(19) United States
(12) Patent Application Publication
(10) Pub. No.: US 2013/0196996 A1
(43) Pub. Date: Aug. 1, 2013

(54) USE OF ISOXAZOLINE COMPOUNDS AND COMPOSITIONS IN BLADDER CANCER

(71) Applicants: John Arthur Taylor, III, Farmington, CT (US); Thais Sielecki-Dzurdz, Kennett Square, PA (US)

(72) Inventors: John Arthur Taylor, III, Farmington, CT (US); Thais Sielecki-Dzurdz, Kennett Square, PA (US)

(73) Assignee: CPSI STOCKHOLDER TRUST, New York, NY (US)

(21) Appl. No.: 13/574,240

(22) PCT Filed: Jan. 19, 2011
(86) PCT No.: PCT/US11/21721

§ 371 Date: Feb. 25, 2013

Related U.S. Application Data
(60) Provisional application No. 61/296,182, filed on Jan. 19, 2010.

(51) Int. Cl. A61K 31/42; A61K 31/4725; A61K 31/4709; A61K 31/422; A61K 31/5377; A61K 31/454

(52) U.S. Cl. CPC A61K 31/42; A61K 31/5377; A61K 31/454; A61K 31/4709; A61K 31/422; A61K 31/4725
USPC 514/236.8; 548/240; 514/378; 544/137; 514/326; 546/209; 514/254.04; 544/367; 546/165; 514/314; 546/146; 514/307

(57) ABSTRACT

One embodiment provides a method, comprising treating or preventing at least one disease selected from the group consisting of bladder cancer, weight loss accompanying bladder cancer, bone pain accompanying bladder cancer, bladder cancer carcinoma in-situ (CIS), TA stage bladder cancer, T1 stage bladder cancer, T2 stage bladder cancer, T3 stage bladder cancer, T4 stage bladder cancer, lymph node metastasis therefrom, solid organ metastasis therefrom, bony metastasis therefrom, cachexia/anorexia associated with bladder cancer, generalized wasting syndrome associated with bladder cancer, or a combination thereof, by administering to a subject in need thereof one or more compounds described herein, prodrug thereof, salt thereof, or a combination thereof.

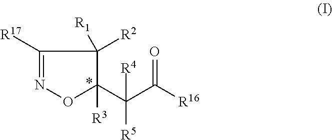

### BACKGROUND

1. Field of the Application

The present application relates to the use of isoxazoline compounds and compositions in bladder cancer.

2. Discussion of the Background

Bladder cancer is the fourth most common solid malignancy in men and the ninth most common solid malignancy in women, representing 7% and 3% of all cancers in men and women respectively. In 2010 there were an estimated 70,500 new cases with 14,680 deaths. Bladder cancer almost never presents as an incidental finding at autopsy, indicating that at some point during the natural history of the tumor it manifests clinically. Stage at diagnosis is fundamental to outcome. High-grade or muscle invasive tumors tend to progress and metastasize with up to 50% of muscle invasive tumors having occult metastatic disease at the time of diagnosis. Invasive and/or metastatic disease carries a relatively poor prognosis with 50% of those with metastatic disease dying within two years of diagnosis. Five year survival rates are as low as 6%. There are no feasible tumor markers capable of stratifying bladder cancer patients with regard to progression, prognosis or treatment. Current therapies for advanced disease are disappointing. Even with aggressive surgical and medical treatment, most patients with advanced bladder cancer ultimately succumb to their disease.

Treatment of high grade bladder cancer has remained essentially unchanged for decades. If disease is found to be superficial, such as CIS or T1, options are for intravesical therapy with limited response or radical surgery. The primary goal of treatment is to prevent the progression to muscle invasion. Although the use of intravesical chemotherapy may increase the time to recurrence, such therapy has little to no impact on disease progression. Bacillus Calmette-Guerin (BCG) intravesical immunotherapy also increases time to first recurrence compared to other chemotherapeutic agents and is currently the standard of care therapy. However, questions remain regarding its value in reducing the progression of the disease. Several meta-analyses have attempted to address this question. In the largest meta-analysis of BCG treatment, the expected relative risk reduction in disease progression is championed at 27%, yet the absolute risk reduction is only 4% (13.8% to 9.8%). Regardless of the small risk reduction in progression, BCG therapy was not shown to have an impact on either disease specific or overall survival.

Standard of care for invasive disease (pT2) remains radical cystectomy with urinary tract reconstruction. Given that bladder cancer is a disease typically seen in the elderly, with average age at diagnosis being 71 and a 3.5 fold increase in the risk of developing invasive disease after the seventh decade of life, existing medical co-morbidities may potentially complicate or preclude the use of aggressive therapy. Thus, the standard treatment for high grade bladder cancer is at best disappointing.

### BRIEF DESCRIPTION OF THE FIGURES

FIG. 1 graphically shows the mean bladder weights in comparative and exemplary groups.

FIG. 2 graphically shows the mean body weights in comparative and exemplary groups.

FIG. 3 graphically shows mean bladder weights in comparative group and exemplary groups.

FIGS. 4A-C show bladder pathology for comparative and exemplary groups. FIG. 4D shows percentage of bladders for comparative and exemplary groups with each pathologic stage. FIG. 4E graphically shows mean percentage of tumors for comparative and exemplary groups.

FIGS. 5A-B show bladder pathology for comparative and exemplary groups.

FIGS. 6A-D show PECAM-1 IHC staining results from a previously published gene deletion model (FIG. 6A); and comparative (FIG. 6B); and exemplary (FIGS. 6C and D) groups.

### DETAILED DESCRIPTION OF SEVERAL EMBODIMENTS

The present application describes the successful use of a class of compounds, described herein, for the prevention and/or treatment of bladder cancer and associated maladies. The present inventors have found that the compounds described herein significantly reduce tumor burden in subjects with bladder cancer. In one embodiment, the compounds described herein significantly reduce the tumor burden with no evident complications or appreciated side effects. In one embodiment, the subjects receiving exemplary embodiments of the compounds described herein appeared healthier than those receiving control compounds and, in every parameter evaluated, displayed a decrease in effective tumor burden.

One embodiment provides a method, comprising treating or preventing at least one disease selected from the group consisting of bladder cancer, weight loss accompanying bladder cancer, bone pain accompanying bladder cancer, bladder cancer carcinoma in-situ (CIS), TA stage bladder cancer, T1 stage bladder cancer, T2 stage bladder cancer, T3 stage bladder cancer, T4 stage bladder cancer, lymph node metastasis therefrom, solid organ metastasis therefrom, bony metastasis therefrom, cachexia/anorexia associated with bladder cancer, generalized wasting syndrome associated with bladder cancer, or a combination thereof, by administering to a subject in need thereof one or more compounds having the following formula (I), prodrug thereof, salt thereof, or a combination thereof, optionally in contact with a pharmaceutically acceptable carrier or excipient:

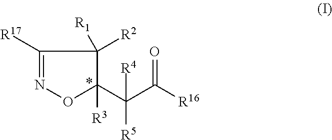

wherein at least the carbon marked “*” is chiral;

wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

wherein R¹⁶ is hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms

and wherein R¹⁷ is an alkyl group, a cycloalkyl group, an alkenyl group, an alkynyl group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

The compound having formula (I) can be in either the R or S configuration. In one embodiment, the compound having formula (I) is in the R configuration. In one embodiment, the compound having formula (I) is in the S configuration. In another embodiment, a mixture of compounds having the R configuration and compounds having the S configuration may be used.

Without wishing to be bound by theory, the present compounds may act through their effect on macrophage migration inhibitory factor. Macrophage migration inhibitory factor (MIF) is a proinflammatory cytokine that has regulatory properties over mediators such as p53 and the retinoblastoma protein (pRb) known to be involved in invasive bladder cancer. MIF activates the MAPK/ERK signaling pathway, which regulates cellular proliferation and survival. MIF has been reported to inhibit p53 tumor suppressor activity via a COX-2/PGE₂ dependant pathway resulting in cell growth and preventing cell regulated apoptosis. It has also been reported to increase cyclin D transcription leading to increased phosphorylation of pRb resulting in cellular proliferation. In addition, MIF has recently been shown to promote survival in fibroblasts via a phosphoinositide-3-kinase (PI3K)/Akt signaling pathway. Recent publications have suggested that MIF expression can lead to increased angiogenesis which is integral to cancer growth, invasion and metastasis.

MIF has been implicated in prostate, lung and breast cancer with overexpression shown to correlate with tumor grade/stage and prognosis. Bladder epithelial cells not only produce MIF but may also display upregulation in response to diverse stimuli. Inhibition of MIF with hyaluronic acid, anti-MIF antibody or MIF anti-sense, was shown to decrease in vitro bladder cancer cell proliferation and cytokine expression. In vivo studies utilizing small interfering RNA (siRNA) transfected cells and inoculation of nude mice showed that inhibition of MIF expression produced tumors with less associated angiogenesis and invasive capacity. Using the N-butyl-N-(4-hydroxybutyl) nitrosamine (BBN) model of bladder cancer we reported that transgenic mice lacking both alleles for MIF developed non-invasive bladder cancers which were associated with decreased tumor associated angiogenesis.

The present application describes, for the first time, the successful use of an oral inhibitor of MIF for the prevention or treatment of bladder cancer. The present inventors have found that the compounds described herein significantly reduce the tumor burden that develops in the BBN mouse model. Further, there were no evident complications or appreciated side effects of drug use. While the results are not identical to those observed in an MIF mouse KO model, they are nevertheless significant, surprising, and unexpected. One would not expect that an oral inhibitor in the form of an oral drug would provide the same efficacy as gene KO. The animals receiving exemplary embodiments of the compounds described herein appeared healthier than those receiving control compounds and, in every parameter evaluated, displayed a decrease in effective tumor burden.

In one embodiment, the methods and compounds described herein provide an effective treatment for subjects with the diagnosis or at risk of any of bladder cancer, weight loss accompanying bladder cancer, bone pain accompanying bladder cancer, bladder cancer carcinoma in-situ (CIS), TA stage bladder cancer, T1 stage bladder cancer, T2 stage bladder cancer, T3 stage bladder cancer, T4 stage bladder cancer, lymph node metastasis therefrom, solid organ metastasis therefrom, bony metastasis therefrom, cachexia/anorexia associated with bladder cancer, generalized wasting syndrome associated with bladder cancer, or a combination thereof.

In one embodiment, the compound having the formula (I) has one of the following formulas, prodrug thereof, salt thereof, or a combination thereof:

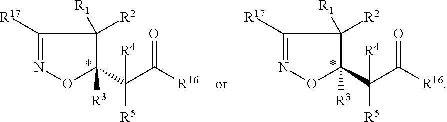

In one embodiment, the compound having formula (I) has the following formula (II), prodrug thereof, salt thereof, or a combination thereof:

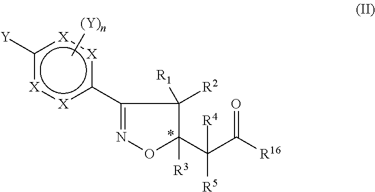

wherein at least the carbon marked “*” is chiral;

wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

wherein each X is independently carbon or nitrogen, wherein when any X is carbon, it comprises a Y substituent, n being an integer of from 1 to 4 and being the number of X's that are carbon;

wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms;

and wherein R¹⁶ is an hydrogen, alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, the compound having the formula (II) has one of the following formulas, prodrug thereof, salt thereof, or a combination thereof:

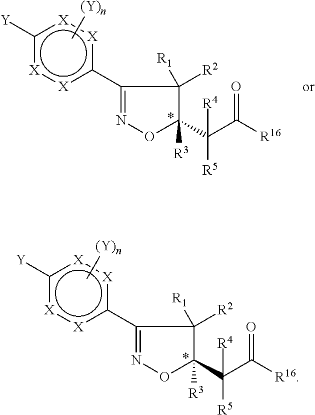

In one embodiment, the compound having formula (I) has one of the following formulas, prodrug thereof, salt thereof, or a combination thereof:

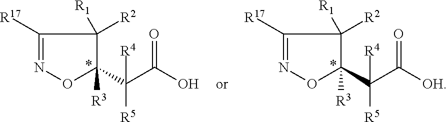

In one embodiment, the compound having the formula (II) has one of the following formulas, prodrug thereof, salt thereof, or a combination thereof:

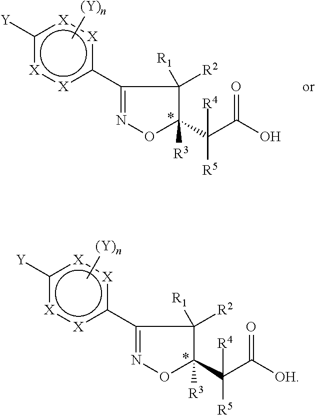

In one embodiment, R¹, R², R³, R⁴, and R⁵ are each independently hydrogen, an alkyl group, a cycloalkyl group, an alkenyl group, an alkynyl group, an oxo group, an aryl group, a heterocyclic group, a heteroaryl group, an aralkyl group, a heteroaralkyl group, an amino group, an alkylamino group, a dialkylamino group, an amidine group, an amide group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, one or both of R⁴ and R⁵ are hydrogen.

In one embodiment, only one of R⁴ and R⁵ is hydrogen.

In one embodiment, R⁴ and R⁵ are independently selected from the group including an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, R¹⁶ is an hydrogen, alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, R¹⁷ is an alkyl group, a cycloalkyl group, an alkenyl group, an alkynyl group, an oxo group, an aryl group, a heterocyclic group, a heteroaryl group, an aralkyl group, a heteroaralkyl group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, an alkyl group is a univalent, acyclic, straight or branched, substituted or unsubstituted, saturated or unsaturated, hydrocarbon radical. In one embodiment, the alkyl group has the general formula (notwithstanding optional unsaturation, substitution or the like) —CₙH₂ₙ₊₁. In one embodiment, n is 1-20 ((C₁-C₂₀) alkyl), which may suitably include C₁, C₂, C₃, C₄, C₅, C₆, C₇, C₈, C_{g}, C₁₀, C₁₁, C₁₂, C₁₃, C₁₄, C₁₅, C₁₆, C₁₇, C₁₈, C₁₉, and C₂₀ alkyl groups. In one embodiment, the alkyl group may be straight or branched, substituted or unsubstituted, saturated or unsaturated, or any combination thereof. In one embodiment, one or more hydrogens may be optionally and independently replaced by one or more substituent groups. In one embodiment, one or more carbon atoms may be optionally and independently replaced with one or more heteroatoms such as O, S, N, B, or any combination thereof. In one embodiment, the alkyl group may contain one or more double bond, one or more triple bond, or any combination thereof. In one embodiment, the alkyl group is attached to the parent structure through one or more independent divalent intervening substituent groups. Some examples of alkyl groups, which are not intended to be limiting, include methyl, ethyl, n-propyl, isopropyl, n-butyl, iso-butyl, secondary-butyl, tertiary-butyl, and the like.

In one embodiment, a cycloalkyl group is a univalent, mono- or polycyclic, substituted or unsubstituted, saturated or unsaturated hydrocarbon radical. In one embodiment, the cycloalkyl group has the general formula (notwithstanding optional unsaturation, substitution, or the like) —CₙH₂ₙ₋₁. In one embodiment, n is 3-20 ((C₃-C₂₀) cycloalkyl), which may suitably include C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ cycloalkyl groups. In one embodiment, the cycloalkyl group is substituted or unsubstituted, saturated or unsaturated, mono-, bi-, tri-, or poly-cyclic, or any combination thereof. In one embodiment, one or more hydrogens may be optionally and independently replaced by one or more substituent groups. In one embodiment, the cycloalkyl group may have one or more sites of unsaturation, e.g., it may contain one or more double bond, one or more triple bond, or any combination thereof. In one embodiment, one or more carbon atoms may be optionally and independently replaced with one or more heteroatoms such as O, S, N, B, or any combination thereof. In one embodiment, the cycloalkyl group is attached to the parent structure through one or more independent divalent intervening substituent groups. Some examples of cycloalkyl groups, which are not intended to be limiting, include cyclopropyl, cyclobutyl, cyclopentyl, cyclohexyl, cycloheptyl, cyclooctyl, cyclononyl, cyclopentenyl, cyclohexenyl, bicyclo[2.2.1]heptanyl, bicyclo[3.2.1]octanyl and bicyclo[5.2.0]nonanyl, and the like.

In one embodiment, an alkenyl group is a univalent, straight or branched, substituted or unsubstituted, unsaturated hydrocarbon radical. In one embodiment, the alkenyl group has the general formula (notwithstanding optional substitution, higher degree of unsaturation, or the like) —CₙH₂ₙ₋₂. In one embodiment, n is 2-20 ((C₂-C₂₀) alkenyl), which may suitably include C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ alkenyl groups. In one embodiment, the alkenyl group may be straight or branched, substituted or unsubstituted, have more than one degree of unsaturation, or any combination thereof. In one embodiment, one or more carbon atoms may be optionally and independently replaced with one or more heteroatoms such as O, S, N, B, or any combination thereof. In one embodiment, the alkenyl group is attached to the parent structure through one or more independent divalent intervening substituent groups. Some examples of alkenyl groups, which are not intended to be limiting, include ethenyl, 1-propenyl, 2-propenyl (allyl), iso-propenyl, 2-methyl-1-propenyl, 1-butenyl, 2-butenyl, alkadienes, alkatrienes, and the like.

In one embodiment, an alkynyl group is a univalent, straight or branched, substituted or unsubstituted, hydrocarbon radical that contains one or more carbon-carbon triple bond. In one embodiment, the alkenyl group has the general formula (notwithstanding optional substitution, higher degree of unsaturation, or the like) —CₙH₂ₙ₋₃. In one embodiment, n is 2-20 ((C₂-C₂₀) alkynyl), which may suitably include C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ alkynyl groups. In one embodiment, the alkynyl group may be straight or branched, substituted or unsubstituted, have more than one degree of unsaturation, or any combination thereof. In one embodiment, one or more carbon atoms may be optionally and independently replaced with one or more heteroatoms such as O, S, N, B, or any combination thereof. In one embodiment, the alkynyl group is attached to the parent structure through one or more independent divalent intervening substituent groups. Some examples of alkynyl groups, which are not intended to be limiting, include alkadiynes, alkatriynes, ethynyl, propynyl, butynyl, and the like.

In one embodiment, an aryl group is a univalent, substituted or unsubstituted, monocyclic or polycyclic aromatic hydrocarbon radical. In one embodiment, an aryl group is a radical which, in accordance with Hückel's theory, includes a cyclic, delocalized (4n+2) pi-electron system. In one embodiment the aryl group is a C₅-C₂₀ aryl group. The C₅-C₂₀ aryl group may suitably include C₅, C₆, C₇, C₈, C₉, C₁₀, C₁₁, C₁₂, C₁₃, C₁₄, C₁₅, C₁₆, C₁₇, C₁₈, C₁₉, and C₂₀ aryl groups. In one embodiment, the aryl group may be substituted or unsubstituted, be substituted with two or more groups that taken together form a cyclic group, or any combination thereof. In one embodiment, the aryl group is attached to the parent structure through one or more independent divalent intervening substituent groups. Some examples of aryl groups, which are not intended to be limiting, include phenyl, naphthyl, tetrahydronaphthyl, phenanthryl, pyrenyl, anthryl, indanyl, chrysyl, and the like.

In one embodiment, a heterocyclic group is a univalent, substituted or unsubstituted, saturated or unsaturated, mono- or polycyclic hydrocarbon radical that contains one or more heteroatoms in one or more of the rings. In one embodiment, the heterocyclic group is a C₃-C₂₀ cyclic group, in which one or more ring carbons is independently replaced with one or more heteroatoms. The C₃-C₂₀ heterocyclic group may suitably include C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ cyclic groups in which one or more ring carbons is independently replaced with one or more heteroatoms. In one embodiment, the heteroatoms are selected from one or more of N, O, or S, or any combination thereof. In one embodiment, the N or S or both may be independently substituted with one or more substituents. In one embodiment, the heterocyclic group is substituted or unsubstituted, saturated or unsaturated, mono-, bi-, tri-, or poly-cyclic, or any combination thereof. In one embodiment, one or more hydrogens may be optionally and independently replaced by one or more substituent groups. In one embodiment, the heterocyclic group may include one or more carbon-carbon double bonds, carbon-carbon triple bonds, carbon-nitrogen double bonds, or any combination thereof. In one embodiment, the heterocyclic group is attached to the parent structure through one or more independent divalent intervening substituent groups. Some examples of heterocyclic groups, which are not intended to be limiting, include azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydrothiadiazinyl, morpholinyl, oxetanyl, tetrahydrodiazinyl, oxazinyl, oxathiazinyl, indolinyl, isoindolinyl, quinuclidinyl, chromanyl, isochromanyl, benzoxazinyl, tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2-pyrazolidin-2-yl, 1,3-pyrazolidin-1-yl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, 1,4-oxazin-2-yl, 1,2,5-oxathiazin-4-yl, and the like

In one embodiment, a heteroaryl group is univalent, substituted or unsubstituted, monocyclic or polycyclic aromatic hydrocarbon radical in which one or more ring carbons is independently replaced with one or more heteroatoms selected from O, S and N. In one embodiment, in addition to said heteroatom, the heteroaryl group may optionally have up to 1, 2, 3, or 4 N atoms in the ring. In one embodiment, the heteroaryl group is an aryl group in which one or more ring carbons are independently replaced with one or more heteroatoms. In one embodiment, a heteroaryl group is an aromatic radical, which contains one or more heteroatoms and which, in accordance with Hückel's theory, includes a cyclic, delocalized (4n+2) pi-electron system. In one embodiment, the heteroaryl group is a C₅-C₂₀ heteroaryl group. The C₅-C₂₀ heteroaryl group may suitably include C₅, C₆, C₇, C₈, C₉, C₁₀, C₁₁, C₁₂, C₁₃, C₁₄, C₁₅, C₁₆, C₁₇, C₁₈, C₁₉, and C₂₀ aryl groups in which one or more than one ring carbon is independently replaced with one or more heteroatoms. In one embodiment, the heteroaryl group may be substituted or unsubstituted, be substituted with two or more groups that taken together form a cyclic group, or any combination thereof. In one embodiment, the heteroaryl group is attached to the parent structure through one or more independent divalent intervening substituent groups. Some examples of heteroaryl groups, which are not intended to be limiting, include heteroaryl group includes pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, and the like.

In one embodiment, an aralkyl group is a univalent radical derived from one or more aryl groups attached to one or more of an alkylene group, cycloalkylene group, alkenylene group, alkynylene group, or combination thereof. The alkylene, cycloalkylene, alkenylene, and alkynylene groups are divalent radicals derived from the removal of hydrogen from the respective alkyl, cycloalkyl, alkenyl, or alkynyl groups. In this context, any combination of aryl group and alkyl, cycloalkyl, alkenyl, or alkynyl group is contemplated. In one embodiment, the aryl group is attached to the parent structure through one or more of the alkylene group, cycloalkylene group, alkenylene group, alkynylene group, or combination thereof as appropriate. In one embodiment, the aralkyl group may be attached to the parent structure through one or more independent divalent intervening substituent groups.

In one embodiment, a heteroaralkyl group is a univalent radical derived from one or more heteroaryl groups attached to one or more of an alkylene group, cycloalkylene group, alkenylene group, alkynylene group, or combination thereof. The alkylene, cycloalkylene, alkenylene, and alkynylene groups are divalent radicals derived from the removal of hydrogen from the respective alkyl, cycloalkyl, alkenyl, or alkynyl groups. In this context, any combination of heteroaryl group and alkyl, cycloalkyl, alkenyl, or alkynyl group is contemplated. In one embodiment, the heteroaryl group is attached to the parent structure through one or more of the alkylene group, cycloalkylene group, alkenylene group, alkynylene group, or combination thereof as appropriate. In one embodiment, the heteroaralkyl group may be attached to the parent structure through one or more independent divalent intervening substituent groups.

In one embodiment, a halo group is a univalent halogen radical or halogen-containing substituent group, e.g., one that is or contains one or more F, Br, Cl, I, or combination thereof. As used herein, the term “halogen” or “halo” includes fluoro, chloro, bromo, or iodo, or fluoride, chloride, bromide or iodide. In one embodiment, a halogen containing substituent group may suitably include a substituent group in which one or more hydrogen atoms are independently replaced with one or more halogens. In one embodiment, the halo group may be attached to the parent structure through one or more independent divalent intervening substituent groups.

In one embodiment, a hydroxy group is a univalent hydroxyl radical (—OH) or hydroxy-containing substituent group, e.g., one that is or contains one or more —OH. As used herein the term, “hydroxy” includes an —OH group. In one embodiment, a hydroxy-containing substituent group may suitably include a substituent group in which one or more hydrogen atoms are independently replaced with one or more —OH groups. In one embodiment, the hydroxyl group may be attached to the parent structure through one or more independent divalent intervening substituent groups.

In one embodiment, an oxo group is a univalent radical that contains an oxygen atom, ═O, doubly bonded to carbon or another element. In one embodiment, the oxo group suitably includes aldehydes, carboxylic acids, ketones, sulfonic acids, amides, esters, and combinations thereof. In one embodiment, the oxo group may be attached to the parent structure through one or more independent divalent intervening substituent groups.

In one embodiment, a mercapto or thiol group is a univalent —SR radical or an —SR-containing group. The R group is suitably chosen from any of the substituent groups. In one embodiment, the mercapto group may be attached to the parent structure through one or more independent divalent intervening substituent groups.

In one embodiment, an amino group is a univalent —NH₂ radical or an —NH₂-containing substituent group. In one embodiment, the amino group may be attached to the parent structure through one or more independent divalent intervening substituent groups.

In one embodiment, an alkylamino group is a univalent —NRH radical or an —NRH-containing substituent group. The R group is suitably chosen from any of the substituent groups. In one embodiment, the alkylamino group may be attached to the parent structure through one or more independent divalent intervening substituent groups.

In one embodiment, a dialkylamino group is a univalent —NRR radical or an —NRR-containing substituent group. The R groups may be the same or different and are suitably and independently chosen from any of the substituent groups. In one embodiment, the dialkylamino group may be attached to the parent structure through one or more independent divalent intervening substituent groups.

In one embodiment, a carbonyl group is a univalent radical that contains a —CR(═O) group. In one embodiment, the carbonyl group suitably includes aldehydes, ketones, and combinations thereof. The R group is suitably chosen from any of the substituent groups. In one embodiment, the carbonyl group may be attached to the parent structure through one or more independent divalent intervening substituent groups.

In one embodiment, a carboxylic acid group is a univalent —C(═O)OH radical or a —C(═O)OH-containing substituent group. In one embodiment, the carboxylic acid group may be attached to the parent structure through one or more independent divalent intervening substituent groups.

In one embodiment, a carboxylate group is a univalent —C(═O)O⁻ anion, —C(═O)OR, or —C(═O)OM, wherein M is a metal cation, or —C(═O)O⁻ anion, —C(═O)OR, or —C(═O)OM-containing substituent group. The R group is suitably chosen from any of the substituent groups. The metal cation is suitably chosen from Li, Na, K, and the like. In one embodiment, the carboxylate group may be attached to the parent structure through one or more independent divalent intervening substituent groups.

In one embodiment, an amidine group is a univalent —C(═NR)NRR radical or a —C(═NR)NRR-containing substituent group. The R groups may be the same or different and are suitably and independently chosen from any of the substituent groups. In one embodiment, the amidine group may be attached to the parent structure through one or more independent divalent intervening substituent groups.

In one embodiment, an amide group is a univalent -E(═O)NRR radical or a -E(═O)NRR-containing substituent group, in which E may be other than carbon, e.g., a chalcogen (e.g., S, Se, Te), or P. In one embodiment, the amide group suitably includes univalent lactams, peptides, phosphoramides, or sulfamides, —S(═O)₂NRR, —P(═O)(OH)NRR, and the like. The R groups may be the same or different and are suitably and independently chosen from any of the substituent groups. In one embodiment, the amide group may be attached to the parent structure through one or more independent divalent intervening substituent groups.

In one embodiment, a carbamoyl group is a univalent —C(═O)NRR radical or a —C(═O)NRR-containing substituent group. The R groups may be the same or different and are suitably and independently chosen from any of the substituent groups. In one embodiment, the carbamoyl group may be attached to the parent structure through one or more independent divalent intervening substituent groups.

In one embodiment, a sulfonyl group is a univalent —S(═O)₂R radical or a —S(═O)₂R-containing substituent group. The R group is suitably chosen from any of the substituent groups. In one embodiment, the sulfonyl group may be attached to the parent structure through one or more independent divalent intervening substituent groups.

In one embodiment, an alkylthio or sulfide group is a univalent —SR radical or an —SR-containing substituent group. The R group is suitably chosen from any of the substituent groups. In one embodiment, the alkylthio group may be attached to the parent structure through one or more independent divalent intervening substituent groups.

In one embodiment, an alkoxy group is a univalent radical derived from an —O-alkyl group. In one embodiment, the alkylthio group may be attached to the parent structure through one or more independent divalent intervening substituent groups.

In one embodiment, an aryloxy group is a univalent radical derived from an —O-aryl group. In one embodiment, the aryloxy group may be attached to the parent structure through one or more independent divalent intervening substituent groups.

In one embodiment, a heteroaryloxy group is a univalent radical derived from an —O-heteroaryl group. In one embodiment, the heteroaryloxy group may be attached to the parent structure through one or more independent divalent intervening substituent groups.

In one embodiment, an aralkoxy group is a univalent radical derived from an —O-aralkyl group. In one embodiment, the aralkoxy group may be attached to the parent structure through one or more independent divalent intervening substituent groups.

In one embodiment, a heteroaralkoxy group is a univalent radical derived from an —O-heteroaryl group. In one embodiment, the heteroaralkoxy group may be attached to the parent structure through one or more independent divalent intervening substituent groups.

In one embodiment, an alkylcarbonyl group is a univalent is radical derived from a -carbonyl-alkyl group. In one embodiment, the alkylcarbonyl group may be attached to the parent structure through one or more independent divalent intervening substituent groups.

In one embodiment, an alkoxycarbonyl group is a univalent radical derived from a -carbonyl-O-alkyl group. In one embodiment, the alkoxycarbonyl group may be attached to the parent structure through one or more independent divalent intervening substituent groups.

In one embodiment, an alkylaminocarbonyl group is a univalent radical derived from a -carbonyl-alkylamino group. In one embodiment, the heteroaralkoxy group may be attached to the parent structure through one or more independent divalent intervening substituent groups.

In one embodiment, a dialkylamino carbonyl group is a univalent radical derived from a -carbonyl-dialkylamino group. In one embodiment, the dialkylamino carbonyl group may be attached to the parent structure through one or more independent divalent intervening substituent groups.

In one embodiment, an arylcarbonyl group is a univalent radical derived from a -carbonyl-aryl group. In one embodiment, the arylcarbonyl group may be attached to the parent structure through one or more independent divalent intervening substituent groups.

In one embodiment, an aryloxycarbonyl group is a univalent radical derived from a -carbonyl-O-aryl group. In one embodiment, the aryloxycarbonyl group may be attached to the parent structure through one or more independent divalent intervening substituent groups.

In one embodiment, an alkylsulfonyl group is a univalent radical derived from a -sulfonyl-alkyl group. In one embodiment, the alkylsulfonyl group may be attached to the parent structure through one or more independent divalent intervening substituent groups.

In one embodiment, an arylsulfonyl group is a univalent radical derived from a -sulfonyl-aryl group. In one embodiment, the arylsulfonyl group may be attached to the parent structure through one or more independent divalent intervening substituent groups.

In one embodiment, a perhaloalkyl group is a univalent radical derived from a completely or substantially completely halogenated alkyl group. In one embodiment, the parhaloalkyl group may be attached to the parent structure through one or more independent divalent intervening substituent groups.

In one embodiment, a perhaloalkoxy group is a univalent radical derived from a completely or substantially completely halogenated alkoxy group. In one embodiment, the arylsulfonyl group may be attached to the parent structure through one or more independent divalent intervening substituent groups.

In one embodiment, a perhalocycloalkyl group is a univalent radical derived from a completely or substantially completely halogenated cycloalkyl group. In one embodiment, the perhalocycloalkyl group may be attached to the parent structure through one or more independent divalent intervening substituent groups.

In one embodiment, a perhaloalkenyl group is a univalent radical derived from a completely or substantially completely halogenated alkenyl group. In one embodiment, the perhaloalkenyl group may be attached to the parent structure through one or more independent divalent intervening substituent groups.

In one embodiment, a perhaloalkynyl group is a univalent radical derived from a completely or substantially completely halogenated alkynyl group. In one embodiment, the perhaloalkynyl group may be attached to the parent structure through one or more independent divalent intervening substituent groups.

In one embodiment, a perhaloaryl group is a univalent radical derived from a completely or substantially completely halogenated aryl group. In one embodiment, the perhaloaryl group may be attached to the parent structure through one or more independent divalent intervening substituent groups.

In one embodiment, a perhaloaralkyl group is a univalent radical derived from a completely or substantially completely halogenated aralkyl group. In one embodiment, the perhaloaralkyl group may be attached to the parent structure through one or more independent divalent intervening substituent groups.

In one embodiment, an alkylcarbonyloxy group is a univalent radical derived from an —O-carbonyl-alkyl group. In one embodiment, the alkylcarbonyloxy group may be attached to the parent structure through one or more independent divalent intervening substituent groups.

In one embodiment, an alkoxycarbonyloxy group is a univalent radical derived from an —O-carbonyl-O-alkyl group. In one embodiment, the alkoxycarbonyloxy group may be attached to the parent structure through one or more independent divalent intervening substituent groups.

In one embodiment, an alkylsulfonyloxy group is a univalent radical derived from an —O-sulfonyl-alkyl group. In one embodiment, the alkylsulfonyloxy group may be attached to the parent structure through one or more independent divalent intervening substituent groups.

In one embodiment, an alkoxysulfonyloxy group is a univalent radical derived from an —O-sulfonyl-O-alkyl group. In one embodiment, the alkoxysulfonyloxy group may be attached to the parent structure through one or more independent divalent intervening substituent groups.

In one embodiment, an arylcarbonyloxy group is a univalent radical derived from an —O-carbonyl-aryl group. In one embodiment, the arylcarbonyloxy group may be attached to the parent structure through one or more independent divalent intervening substituent groups.

In one embodiment, an aryloxycarbonyloxy group is a univalent radical derived from an —O-carbonyl-O-aryl group. In one embodiment, the aryloxycarbonyloxy group may be attached to the parent structure through one or more independent divalent intervening substituent groups.

In one embodiment, an arylsulfonyloxy group is a univalent radical derived from an —O-sulfonyl-aryl group. In one embodiment, the arylsulfonyloxy group may be attached to the parent structure through one or more independent divalent intervening substituent groups.

In one embodiment, an aryloxysulfonyloxy group is a univalent radical derived from an —O-sulfonyl-O-aryl group. In one embodiment, the aryloxysulfonyloxy group may be attached to the parent structure through one or more independent divalent intervening substituent groups.

In one embodiment, referring to two groups taken together to form a cyclic group, the cyclic group may be suitably derived from a divalent cycloalkylene group or divalent heterocyclic group. The divalent cycloalkylene and heterocyclic groups may be suitably derived from the respective cycloalkyl or heterocyclic groups.

In one embodiment, referring to two groups taken together to form an aryl group, the aryl group may be suitably derived from a divalent arylene group or divalent heteroarlyene group. The divalent arylene and heteroarylene groups may be suitably derived from the respective aryl or heteroaryl groups.

In one embodiment, referring to the replacement of one or more than one atom in each group with one or more heteroatoms, the heteroatoms may be suitably chosen from N, O, P, S, B, or any combination thereof as appropriate.

In one embodiment, the structure

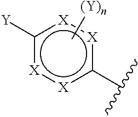

may have one of the following three structures:

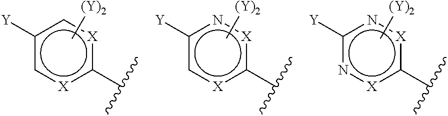

wherein each X is independently carbon or nitrogen, and wherein X is carbon, it independently comprises a Y substituent. In the three structures shown above, in one embodiment, the X's may be carbon, each carbon independently comprising a Y substituent.

In one embodiment, the structure

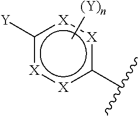

may be one of the following structures:

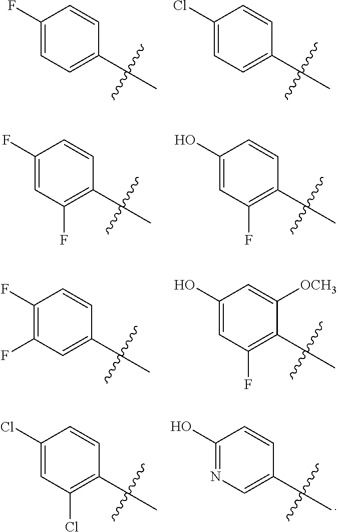

In one embodiment, Y may be an alkyl group, a cycloalkyl group, a halo group, a perfluoroalkyl group, a perfluoroalkoxy group, an alkenyl group, an alkynyl group, a hydroxy group, an oxo group, a mercapto group, an alkylthio group, an alkoxy group, an aryl group, a heteraryl group, an aryloxy group, a heteroaryloxy group, an aralkyl group, a heteroaralkyl group, an aralkoxy group, a heteroaralkoxy group, an HO—(C═O)— group, an amino group, an alkylamino group, a dialkylamino group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, or an arylsulfonyl group, or have the following structure:

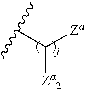

in which each Z^{a} is independently either hydrogen, hydroxyl, halogen, or a substituent group; and

“j” is independently either zero or an integer from one to four.

In one embodiment, the structure:

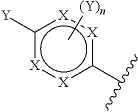

has the following structure:

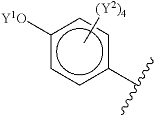

in which each Y¹ is independently a hydrogen or (C₁-C₆)alkyl; and

each Y² is independently a Y¹, hydroxyl group, halo group, —N₃, —CN, —SH, or —N(Y¹)₂.

In one embodiment, the compound having formula (I) has the following structure:

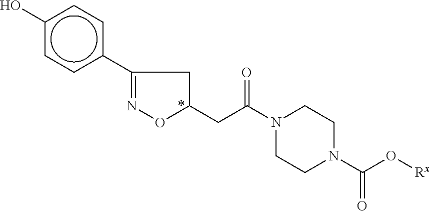

wherein R^{X} is a (C₁-C₆)alkyl, (C₂-C₆)alkenyl, (C₂-C₆)alkynyl, phenyl, (C₅-C₁₄)aryl, (C₄-C₁₄)heteroaryl, (C₂-C₁₄)heterocyclic or (C₃-C₁₀)cycloalkyl group.

In one embodiment, the compound having formula (I) has the following structure:

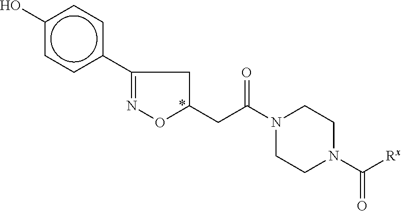

wherein R^{X} is a (C₁-C₆)alkyl, (C₂-C₆)alkenyl, (C₂-C₆)alkynyl, phenyl, (C₅-C₁₄)aryl, (C₄-C₁₄)heteroaryl, (C₂-C₁₄)heterocyclic or (C₃-C₁₀)cycloalkyl group.

In one embodiment, the compound having formula (I) has the following structure:

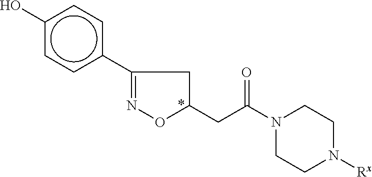

wherein R^{X} is a sulfonyl, carbonyl, (C₁-C₆)alkylsulfonyl, (C₁-C₆)alkylcarbonyl, (C₅-C₁₄)arylsulfonyl, (C₅-C₁₄)arylcarbonyl, (C₁-C₆)alkyl, (C₂-C₆)alkenyl, (C₂-C₆)alkynyl, phenyl, (C₅-C₁₄)aryl, (C₄-C₁₄)heteroaryl, (C₂-C₁₄)heterocyclic or (C₃-C₁₀)cycloalkyl group.

In one embodiment, the compound having formula (I) has the following structure:

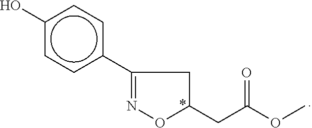

In one embodiment, the compound having formula (I) has the following structure:

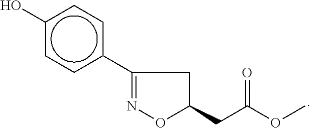

In one embodiment, the compound having formula (I) has the following structure:

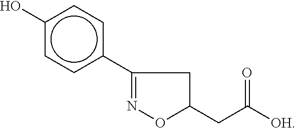

In one embodiment, the compound having formula (I) has the following structure:

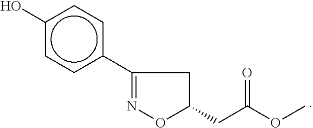

In one embodiment, the compound having formula (I) has the following structure:

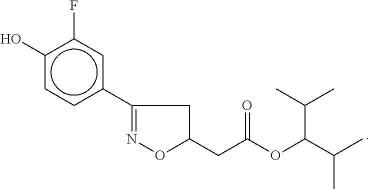

In one embodiment, the compound having formula (I) has the following structure:

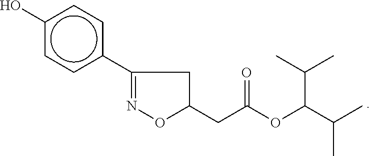

In one embodiment, the compound having formula (I) has the following structure:

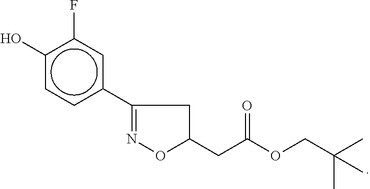

In one embodiment, the compound having formula (I) has the following structure:

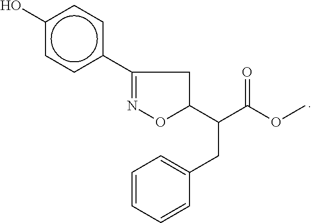

In one embodiment, the compound having formula (I) has the following structure:

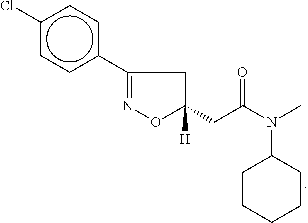

In one embodiment, the compound having formula (I) has the following structure:

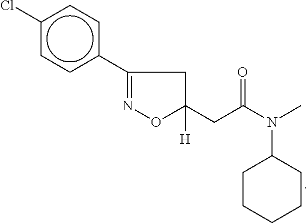

In one embodiment, the compound having formula (I) has the following structure:

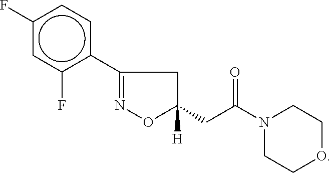

In one embodiment, the compound having formula (I) has the following structure:

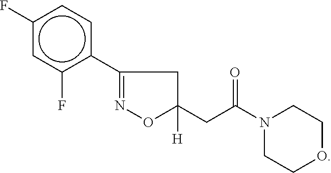

In one embodiment, R¹⁶ can be one of the following structures:

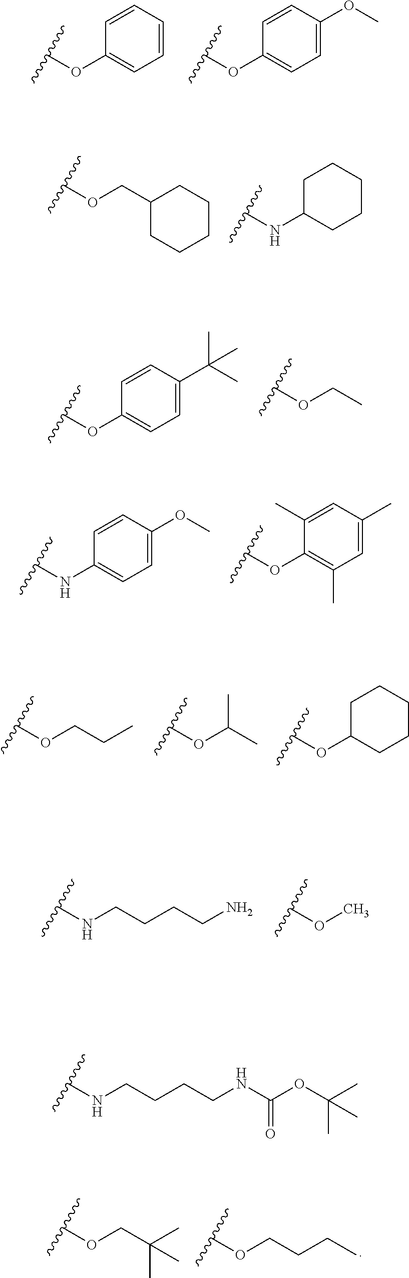

In one embodiment, the substituent groups described herein may be suitably and independently chosen from one or more of a hydrogen, an azido group, a carbamido group, a carbazoyl group, a cyanato group, a cyano group, an isocyanato group, an isocyano group, a hydroxamino group, a guanidino group, a guanyl group, an imino group, a nitro group, a phospho group, a phosphate group, a phosphine group, a sulfo group, a sulfate group, a sulfonyl group, a carbonyl group, a carboxylic acid group, a carboxylate group, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, an alkylcarbonyloxy group, an alkoxycarbonyloxy group, an alkylsulfonyloxy group, an alkoxysulfonyloxy group, an arylcarbonyloxy group, an aryloxycarbonyloxy group, an arylsulfonyloxy group, an aryloxysulfonyloxy group, an a perhaloalkyl group, a perhaloalkoxy group, a perhalocycloalkyl group, a perhaloalkenyl group, a perhaloalkynyl group, a perhaloaryl group, a perhaloaralkyl group, or combination thereof. Univalent residues or divalent intervening residues of any substituent group or combination thereof may be suitably used as appropriate.

In one embodiment, the divalant intervening substituent groups may be suitably and independently chosen from one or more of an azo group, an azino group, an azoxy group, a carbonyl group, a dioyl group, a diazoamino group, a disulfinyl group, a dithio group, an oxy group, a hydrazo group, an oxalyl group, a sulfonyl group, a thiocarbonyl group, a thionyl group, a phosphono ester group, a carboxylate group, a thio group; divalent residues of one or more of the following groups: an alkyl group, a cycloalkyl group, an alkenyl group, an alkynyl group, an alkylthio group, an alkyloxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, an alkylcarbonyloxy group, an alkoxycarbonyloxy group, an alkylsulfonyloxy group, an alkoxysulfonyloxy group, an arylcarbonyloxy group, an aryloxycarbonyloxy group, an arylsulfonyloxy group, an aryloxysulfonyloxy group, an a perhaloalkyl group, a perhaloalkoxy group, a perhalocycloalkyl group, a perhaloalkenyl group, a perhaloalkynyl group, a perhaloaryl group, a perhaloaralkyl group, combination thereof; or combination thereof.

In one embodiment, when the compound having formula (I) has the following formula (II), prodrug thereof, salt thereof, or a combination thereof:

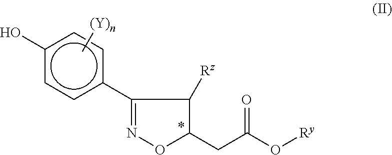

and each Y is independently H or F, and R^{y} is a straight or branched C₁-C₁₀ alkyl, then Rz is not phenyl or hydrogen.

All of the compounds described herein may be easily prepared without undue experimentation by known methods given the teachings herein and the knowledge available to one of ordinary skill in organic chemical syntheses. Examples of preparation methods may be found in, for example, U.S. application Ser. Nos. 10/164,630, filed Jun. 10, 2002; 10/927,494, filed Aug. 27, 2004; 11/090,128, filed Mar. 28, 2005; and 61/264,406, filed Nov. 25, 2009 (now PCT/US10/58135, filed Nov. 26, 2010), which are independently incorporated herein by reference.

In one embodiment, the compounds may be present in a composition, or optionally in combination with a pharmaceutically acceptable carrier or excipient, as mixtures of diastereomers. In another embodiment, they may be present, optionally in combination with a pharmaceutically acceptable carrier or excipient, as a single diastereomer. In one embodiment, one diastereomer may be present in an amount greater than 50 mol % relative to other diastereomers present in the composition, if any. This range may suitably include greater than 50, 51, 52, 53, 54, 55, 56, 57, 58, 59, 60, 65, 70, 75, 80, 85, 90, 91, 92, 93, 94, 95, 96, 97, 98, 99, 99.0, 99.1, 99.2, 99.3, 99.4, 99.5, 99.6, 99.7, 99.8, 99.9, and 100 mol % of one diastereomer relative to any other diastereomer.

In one embodiment, if present in a mixture of diastereomeric pairs of compounds, the diastereomer:diastereomer ratio may suitably range from greater than 50:less than 50 to 100:0. This range of ratios includes all values and subranges therebetween, including greater than 50, 51, 52, 53, 54, 55, 56, 57, 58, 59, 60, 61, 62, 63, 64, 65, 66, 67, 68, 69, 70 71, 72, 73, 74, 75, 76, 77, 78, 79, 80, 81, 82, 83, 84, 85, 86, 87, 88, 89, 90, 91, 92, 93, 94, 95, 96, 97, 98, 99, 100: less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.

In one embodiment, the mixtures of diastereomers can be separated without undue experimentation using separation methods well known in the art, such as recrystallization or chromatography or a combination thereof. The recrystallizations can accomplished in organic solvents such as, but not limited to, pentane, hexane, cyclohexane, toluene, benzene, chlorobutane, dichloromethane, diethyl ether, tetrahydrofuran, dimethoxyethane, acetonitrile, methanol, ethanol or butanol or a combination of organic solvents with or without water. The chromatography can be accomplished with a silica gel or alumina solid phase, eluting with mixtures of organic solvents, with or without acidic or basic modifiers, such as triethylamine, aqueous ammonia, acetic acid or aqueous hydrochloric acid.

In one embodiment, at least one compound having formula (I), salt thereof, prodrug thereof, or combination thereof, may be suitably used in contact with at least one pharmaceutically acceptable carrier or excipient, for use in the methods described herein as a pharmaceutical composition.

In one embodiment, at least one compound having formula (I), salt thereof, prodrug thereof, or combination thereof, optionally in contact with at least one pharmaceutically acceptable carrier or excipient, may be used for the preparation of a medicament for the methods described herein.

In one embodiment, pharmaceutically acceptable means a material that is compatible with the other ingredients of the composition without rendering the composition unsuitable for its intended purpose, and is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers or excipients include, without limitation, any of the standard pharmaceutical carriers or excipients such as phosphate buffered saline solutions, water, emulsions such as oil/water emulsions, microemulsions, and the like.

The compounds and compositions described herein can be formulated without undue experimentation for administration to a mammal, including humans, as appropriate for the particular application. The pharmaceutical compositions may be manufactured without undue experimentation in a manner that is itself known, e.g., by means of conventional mixing, dissolving, dragee-making, levitating, emulsifying, encapsulating, entrapping, spray-drying, or lyophilizing processes, or any combination thereof.

Suitable routes of administration may include, for example, oral, lingual, sublingual, rectal, transmucosal, nasal, buccal, intrabuccal, intravaginal, or intestinal administration; intravesicular; intraurethral; topical administration; transdermal administration; administration by inhalation; parenteral delivery, non-parenteral delivery, including intramuscular, subcutaneous, intramedullary injections, as well as intrathecal, direct intraventricular, intravenous, intraperitoneal, intranasal, or intraocular injections, and optionally in a depot or sustained release formulation. Furthermore, one may administer the compound in a targeted drug delivery system, for example in a liposome. Combinations of administrative routes are possible.

Proper dosages of the compounds and compositions can be determined without undue experimentation using standard dose-response protocols. In one embodiment, the dosage of the compound, salt thereof, or a combination thereof, or pharmaceutical composition, may vary from about 0.001 μg/kg to about 1000 mg/kg. This includes all values and subranges therebetween, including 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1 μg/kg, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1 2, 3, 4, 5, 6, 7, 8, 9, 10, 11, 12, 13, 14, 15, 16, 17, 18, 19, 20, 21, 22, 23, 24, 25, 50, 75, 100, 200, 300, 400, 500, 600, 700, 800, 900, and 1000 mg/kg, and any combination thereof.

The frequency of administration of the compounds and compositions can be determined without undue experimentation using standard dose-response protocols. In one embodiment, the frequency of administration of the compound, salt thereof, or a combination thereof, or pharmaceutical composition, may vary from about 0.5 hours to one month. This range includes all values and subranges therebetween, including about 0.5, 0.75, 1, 2, 3, 4, 5, 6, 7, 8, 9, 10, 12, 14, 16, 18, 20, 24 hours, 1, 2, 3, 4, 5, 6, 7, 8, 9, 10, 12, 14, 16, 18, 20, 24, 28, 30, 31 days, and one month, or any combination thereof. The administration can be suitably adjusted in the case a controlled or sustained release formulation is used. The compounds and compositions for administration can be made without undue experimentation by means well known in the art, for example with pharmaceutically acceptable carrier or excipient for example an inert diluent, solvent, suspending agent or the like. The compositions may be enclosed in gelatin capsules or compressed into tablets. In one embodiment, the pharmaceutical compositions may be incorporated with a carrier or excipient and used in the form of tablets, troches, capsules, elixirs, suspensions, syrups, wafers, chewing gums and the like.

In one embodiment, the compounds and/or compositions are administered orally.

Tablets, pills, capsules, troches and the like may also contain pharmaceutically acceptable carriers or excipients for example binders, additives, disintegrating agents, lubricants, sweetening agents, and flavoring agents. Some examples of binders include microcrystalline cellulose, gum tragacanth or gelatin. Examples of additives include starch or lactose. Some examples of disintegrating agents include alginic acid, cornstarch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compounds can easily be administered parenterally such as for example, by intravenous, intramuscular, intrathecal or subcutaneous injection. Parenteral administration can be accomplished by incorporating the compounds into a solution or suspension. Such solutions or suspensions may also include sterile diluents such as water, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the compound, in a pharmaceutical composition, into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Some examples of transdermal formulations include patches (such as the well-known nicotine patch), iontophoretic devices, microneedles, ointments, creams, gels, salves and the like.

The compounds can also be prepared for nasal administration. As used herein, nasal administration includes administering the compound to the mucous membranes of the nasal passage or nasal cavity of the subject. Pharmaceutical compositions for nasal administration of the compound include therapeutically effective amounts of the compound prepared by well-known methods to be administered, for example, as a nasal spray, nasal drop, suspension, gel, ointment, cream or powder. Administration of the compound may also take place using a nasal tampon or nasal sponge.

The compounds may be administered per se (neat) or in the form of a pharmaceutically acceptable salt. When used in medicine, the salts should be both pharmacologically and pharmaceutically acceptable, but non-pharmaceutically acceptable salts may conveniently be used to prepare the free active compound or pharmaceutically acceptable salts thereof. Pharmacologically and pharmaceutically acceptable salts include, but are not limited to, those prepared from the following acids: hydrochloric, hydrobromic, sulphuric, nitric, phosphoric, maleic, acetic, salicyclic, p-toluenesulfonic, tartaric, citric, methanesulphonic, formic, malonic, succinic, naphthalene-2-sulphonic, and benzenesulphonic. Also, pharmaceutically acceptable salts can be prepared as alkaline metal or alkaline earth salts, such as sodium, potassium or calcium salts of the carboxylic acid group. Both simple and complex salts are possible.

The compositions may also suitably include one or more preservatives, anti-oxidants, or the like.

The compounds can be administered orally. Thus, in any of the methods herein, the pharmaceutical composition can be administered orally. Alternatively, the pharmaceutical composition can be administered parenterally.

Some examples of techniques for the formulation and administration of the compounds may be found in Remington: The Science and Practice of Pharmacy, Lippincott Williams & Wilkins Publishing Co., 21^{st} addition, incorporated herein by reference.

In one embodiment, the compound may be chemically modified for administration in the form of a prodrug. As is known in the art, prodrugs liberate the active compound in vivo by enzymatic or chemical processes, and their preparation can be carried out without undue experimentation given the teachings herein and the knowledge available to one of skill in the art.

Any combination of one or more compounds, salts, prodrugs, metabolites, isotopically-labeled compounds, tautomers, isomers, enantiomers, diastereomers, and/or atropisomers is possible in the pharmaceutical composition.

In one embodiment, the compounds may be delivered using a sustained-release system, such as semipermeable matrices of solid hydrophobic polymers containing the therapeutic agent. Various forms of sustained-release materials are well known by those skilled in the art and can be prepared without undue experimentation. Sustained-release capsules may, depending on their chemical nature, release the compounds for a few hours, a few days, a few weeks to up to over 100 days. Depending on the chemical nature and the biological stability of the compound, additional strategies for stabilization may be employed.

In one embodiment, the pharmaceutical compositions contain the compound having formula (I) in an effective amount to achieve their intended purpose. In one embodiment, an effective amount means an amount sufficient to prevent or treat the disease. In one embodiment, to treat means to reduce the development of, inhibit the progression of, or ameliorate the symptoms of a disease in the subject being treated. In one embodiment, to treat means to reduce or inhibit the metastasis of a disease in the subject being treated. In one embodiment, to prevent means to administer prophylactically, e.g., in the case wherein in the opinion of the attending physician the subject's background, heredity, environment, occupational history, or the like, give rise to an expectation or increased probability that that subject is at risk of contracting the disease, even though at the time of diagnosis or administration that subject either does not yet have the disease or is asymptomatic of the disease. In one embodiment, prevention reduces the likelihood of an individual contracting the disease. In another embodiment, prevention reduces the number of individuals that contract the disease in a population.

Each of the prevention and/or treatment of the disease, for example, bladder cancer, weight loss accompanying bladder cancer, bone pain accompanying bladder cancer, bladder cancer carcinoma in-situ (CIS), TA stage bladder cancer, T1 stage bladder cancer, T2 stage bladder cancer, T3 stage bladder cancer, T4 stage bladder cancer, lymph node metastasis therefrom, solid organ metastasis therefrom, bony metastasis therefrom, cachexia/anorexia associated with bladder cancer, generalized wasting syndrome associated with bladder cancer, or a combination thereof, by administering to a subject in need thereof one or more compounds having the formula (I), prodrug thereof, salt thereof, or a combination thereof, optionally in contact with a pharmaceutically acceptable carrier or excipient; the identification of a subject and determination of that subject's need for the treatment or prevention of bladder cancer, weight loss accompanying bladder cancer, bone pain accompanying bladder cancer, bladder cancer carcinoma in-situ (CIS), TA stage bladder cancer, T1 stage bladder cancer, T2 stage bladder cancer, T3 stage bladder cancer, T4 stage bladder cancer, lymph node metastasis therefrom, solid organ metastasis therefrom, bony metastasis therefrom, cachexia/anorexia associated with bladder cancer, generalized wasting syndrome associated with bladder cancer, or a combination thereof, by administering to a subject in need thereof one or more compounds having the formula (I), prodrug thereof, salt thereof, or a combination thereof, optionally in contact with a pharmaceutically acceptable carrier or excipient; and the determination of the effective amount of the compound having formula (I) and route of administration can be carried out without undue experimentation by the skilled artisan in light of the detailed disclosure herein.

One embodiment provides a method, comprising treating at least one disease selected from the group consisting of bladder cancer, weight loss accompanying bladder cancer, bone pain accompanying bladder cancer, bladder cancer carcinoma in-situ (CIS), TA stage bladder cancer, T1 stage bladder cancer, T2 stage bladder cancer, T3 stage bladder cancer, T4 stage bladder cancer, lymph node metastasis therefrom, solid organ metastasis therefrom, bony metastasis therefrom, cachexia/anorexia associated with bladder cancer, generalized wasting syndrome associated with bladder cancer, or a combination thereof, by administering to a subject in need thereof one or more compounds having formula (I), prodrug thereof, salt thereof, or a combination thereof, optionally in contact with a pharmaceutically acceptable carrier or excipient.

One embodiment provides a method, comprising preventing at least one disease selected from the group consisting of bladder cancer, weight loss accompanying bladder cancer, bone pain accompanying bladder cancer, bladder cancer carcinoma in-situ (CIS), TA stage bladder cancer, T1 stage bladder cancer, T2 stage bladder cancer, T3 stage bladder cancer, T4 stage bladder cancer, lymph node metastasis therefrom, solid organ metastasis therefrom, bony metastasis therefrom, cachexia/anorexia associated with bladder cancer, generalized wasting syndrome associated with bladder cancer, or a combination thereof, by administering to a subject in need thereof one or more compounds having formula (I), prodrug thereof, salt thereof, or a combination thereof, optionally in contact with a pharmaceutically acceptable carrier or excipient.

In one embodiment, the subject is a mammal. In one embodiment, the mammal is a human, an animal, a domesticated animal, a dog, a cat, a livestock, a cow, a pig, a horse, and the like. In one embodiment, the subject in need of such treatment and/or prevention is known or suspected to suffer from or at risk of contracting at least one disease selected from the group consisting of bladder cancer, weight loss accompanying bladder cancer, bone pain accompanying bladder cancer, bladder cancer carcinoma in-situ (CIS), TA stage bladder cancer, T1 stage bladder cancer, T2 stage bladder cancer, T3 stage bladder cancer, T4 stage bladder cancer, lymph node metastasis therefrom, solid organ metastasis therefrom, bony metastasis therefrom, cachexia/anorexia associated with bladder cancer, generalized wasting syndrome associated with bladder cancer, or a combination thereof.

Some examples of compounds which have been prepared and which may be suitably used in accordance with the present description may be found in the table below, which is not intended to be limiting. The compound having formula (I) can have any of the following structures:

| Structure | MF | MW |
|---|---|---|
| 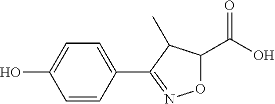 [SMILES: CC1C(c2ccc(O)cc2)=NOC1C(=O)O] | C11H11NO4 | 221.21 |
| 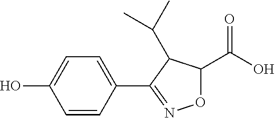 [SMILES: CC(C)C1C(c2ccc(O)cc2)=NOC1C(=O)O] | C13H15NO4 | 249.29 |
| 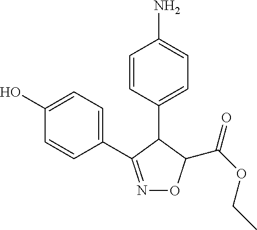 [SMILES: CCOC(=O)C1ON=C(c2ccc(O)cc2)C1c1ccc(N)cc1] | C18H18N2O4 | 326.36 |
| 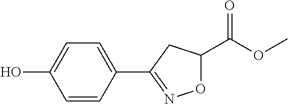 [SMILES: COC(=O)C1CC(c2ccc(O)cc2)=NO1] | C11H11NO4 | 221.21 |
| 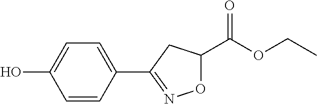 [SMILES: CCOC(=O)C1CC(c2ccc(O)cc2)=NO1] | C12H13NO4 | 235.24 |
| 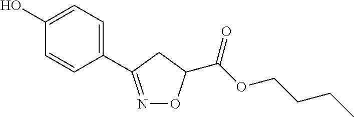 [SMILES: CCCCOC(=O)C1CC(c2ccc(O)cc2)=NO1] | C14H17NO4 | 263.3 |
| 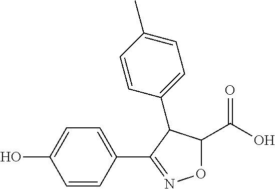 [SMILES: Cc1ccc(C2C(c3ccc(O)cc3)=NOC2C(=O)O)cc1] | C17H15NO4 | 297.31 |
| 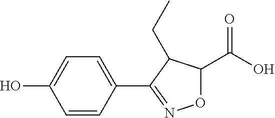 [SMILES: CCC1C(c2ccc(O)cc2)=NOC1C(=O)O] | C12H13NO4 | 235.24 |
| 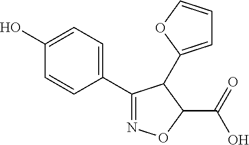 [SMILES: O=C(O)C1ON=C(c2ccc(O)cc2)C1c1ccco1] | C14H11NO5 | 273.24 |
| 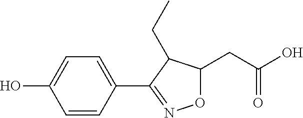 [SMILES: CCC1C(c2ccc(O)cc2)=NOC1CC(=O)O] | C13H15NO4 | 249.27 |
| 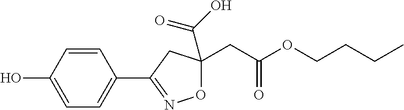 [SMILES: CCCCOC(=O)CC1(C(=O)O)CC(c2ccc(O)cc2)=NO1] | C16H19NO6 | 321.33 |
| 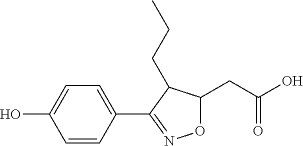 [SMILES: CCCC1C(c2ccc(O)cc2)=NOC1CC(=O)O] | C14H17NO4 | 263.3 |
| 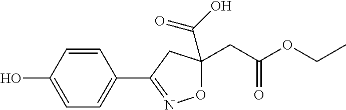 [SMILES: CCOC(=O)CC1(C(=O)O)CC(c2ccc(O)cc2)=NO1] | C14H15NO6 | 293.27 |
| 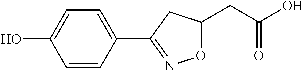 [SMILES: O=C(O)CC1CC(c2ccc(O)cc2)=NO1] | C11H11NO4 | 221.21 |
| 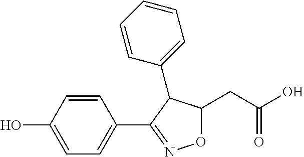 [SMILES: O=C(O)CC1ON=C(c2ccc(O)cc2)C1c1ccccc1] | C17H15NO4 | 297.31 |
| 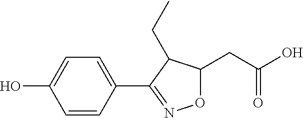 [SMILES: CCC1C(c2ccc(O)cc2)=NOC1CC(=O)O] | C13H15NO4 | 249.29 |
| 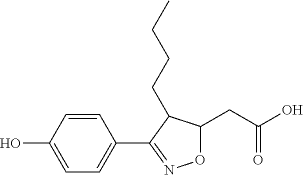 [SMILES: CCCCC1C(c2ccc(O)cc2)=NOC1CC(=O)O] | C15H19NO4 | 277.32 |
| 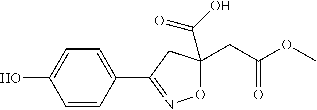 [SMILES: COC(=O)CC1(C(=O)O)CC(c2ccc(O)cc2)=NO1] | C13H13NO6 | 279.25 |
| 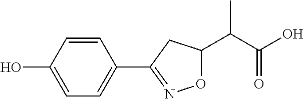 [SMILES: CC(C(=O)O)C1CC(c2ccc(O)cc2)=NO1] | C12H13NO4 | 235.24 |
| 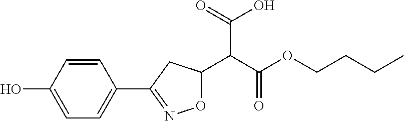 [SMILES: CCCCOC(=O)C(C(=O)O)C1CC(c2ccc(O)cc2)=NO1] | C16H19NO6 | 321.33 |
| 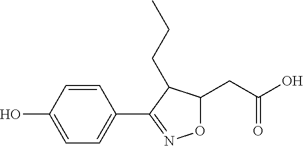 [SMILES: CCCC1C(c2ccc(O)cc2)=NOC1CC(=O)O] | C14H17NO4 | 263.29 |
| 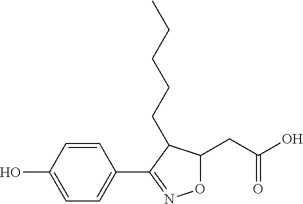 [SMILES: CCCCCC1C(c2ccc(O)cc2)=NOC1CC(=O)O] | C16H21NO4 | 291.34 |
| 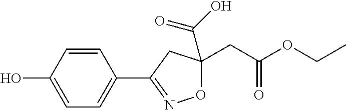 [SMILES: CCOC(=O)CC1(C(=O)O)CC(c2ccc(O)cc2)=NO1] | C14H15NO6 | 293.27 |
| 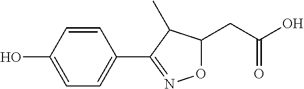 [SMILES: CC1C(c2ccc(O)cc2)=NOC1CC(=O)O] | C12H13NO4 | 235.24 |
| 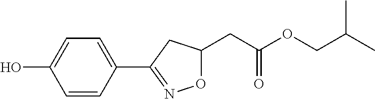 [SMILES: CC(C)COC(=O)CC1CC(c2ccc(O)cc2)=NO1] | C15H19NO4 | 277.32 |
| 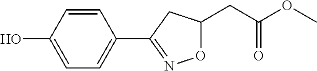 [SMILES: COC(=O)CC1CC(c2ccc(O)cc2)=NO1] | C12H13NO4 | 235.24 |
| 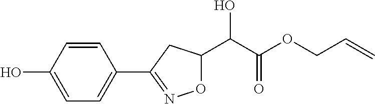 [SMILES: C=CCOC(=O)C(O)C1CC(c2ccc(O)cc2)=NO1] | C14H15NO5 | 277.27 |
| 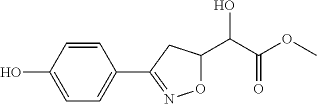 [SMILES: COC(=O)C(O)C1CC(c2ccc(O)cc2)=NO1] | C12H13NO5 | 251.24 |
| 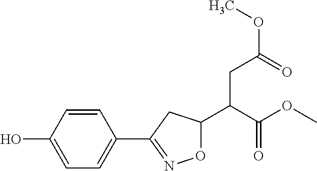 [SMILES: COC(=O)CC(C(=O)OC)C1CC(c2ccc(O)cc2)=NO1] | C15H17NO6 | 307.31 |
| 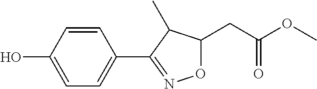 [SMILES: COC(=O)CC1ON=C(c2ccc(O)cc2)C1C] | C13H15NO4 | 249.26 |
| 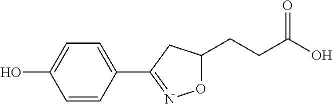 [SMILES: O=C(O)CCC1CC(c2ccc(O)cc2)=NO1] | C12H13NO4 | 235.24 |
| 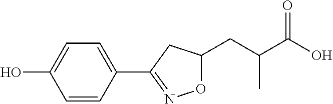 [SMILES: CC(CC1CC(c2ccc(O)cc2)=NO1)C(=O)O] | C13H15NO4 | 249.27 |
| 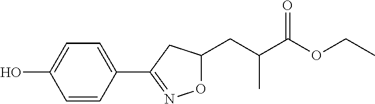 [SMILES: CCOC(=O)C(C)CC1CC(c2ccc(O)cc2)=NO1] | C15H19NO4 | 277.32 |
| 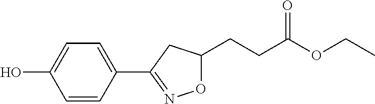 [SMILES: CCOC(=O)CCC1CC(c2ccc(O)cc2)=NO1] | C14H17NO4 | 263.3 |
| 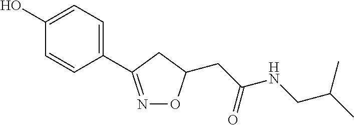 [SMILES: CC(C)CNC(=O)CC1CC(c2ccc(O)cc2)=NO1] | C15H20N2O3 | 276.33 |
| 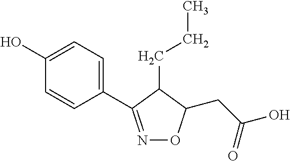 [SMILES: CCCC1C(c2ccc(O)cc2)=NOC1CC(=O)O] | C14H17NO4 | 263.29 |
| 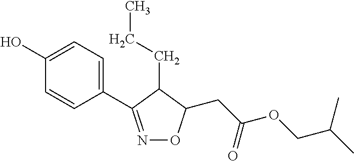 [SMILES: CCCC1C(c2ccc(O)cc2)=NOC1CC(=O)OCC(C)C] | C19H25NO4 | 319.4 |
| 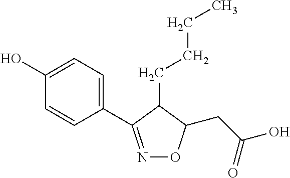 [SMILES: CCCCC1C(c2ccc(O)cc2)=NOC1CC(=O)O] | C15H19NO4 | 277.32 |
| 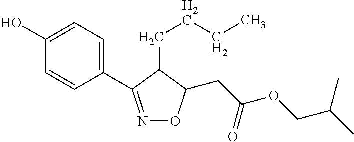 [SMILES: CCCCC1C(c2ccc(O)cc2)=NOC1CC(=O)OCC(C)C] | C19H27NO4 | 333.42 |
| 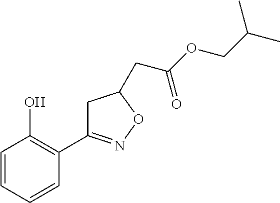 [SMILES: CC(C)COC(=O)CC1CC(c2ccccc2O)=NO1] | C15H19NO4 | 277.32 |
| 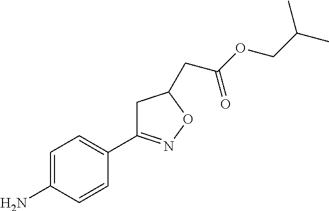 [SMILES: CC(C)COC(=O)CC1CC(c2ccc(N)cc2)=NO1] | C15H20N2O3 | 276.33 |
| 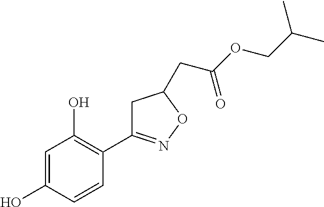 [SMILES: CC(C)COC(=O)CC1CC(c2ccc(O)cc2O)=NO1] | C15H19NO5 | 293.32 |
| 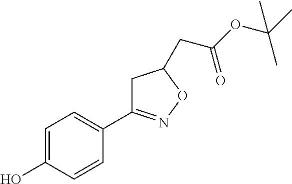 [SMILES: CC(C)(C)OC(=O)CC1CC(c2ccc(O)cc2)=NO1] | C15H19NO4 | 277.32 |
| 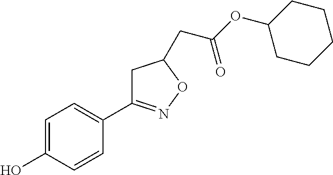 [SMILES: O=C(CC1CC(c2ccc(O)cc2)=NO1)OC1CCCCC1] | C17H21NO4 | 303.35 |
| 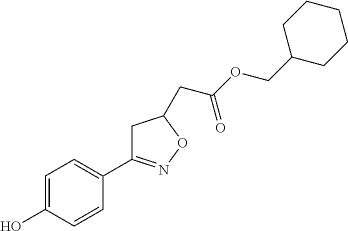 [SMILES: O=C(CC1CC(c2ccc(O)cc2)=NO1)OCC1CCCCC1] | C18H23NO4 | 317.38 |
| 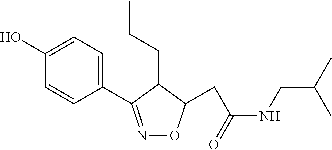 [SMILES: CCCC1C(c2ccc(O)cc2)=NOC1CC(=O)NCC(C)C] | C18H26N2O3 | 318.41 |
| 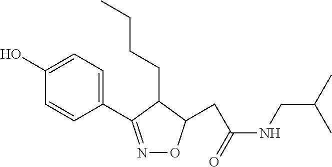 [SMILES: CCCCC1C(c2ccc(O)cc2)=NOC1CC(=O)NCC(C)C] | C19H28N2O3 | 332.44 |
| 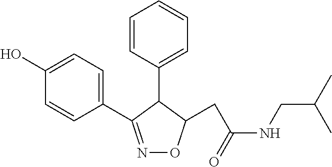 [SMILES: CC(C)CNC(=O)CC1ON=C(c2ccc(O)cc2)C1c1ccccc1] | C21H24N2O3 | 352.43 |
| 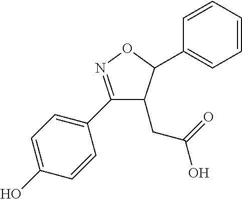 [SMILES: O=C(O)CC1C(c2ccc(O)cc2)=NOC1c1ccccc1] | C17H15NO4 | 297.31 |
| 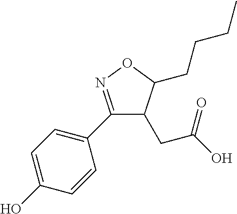 [SMILES: CCCCC1ON=C(c2ccc(O)cc2)C1CC(=O)O] | C15H19NO4 | 277.32 |
| 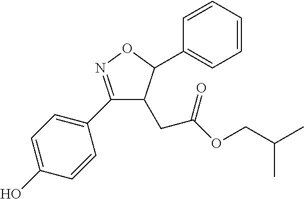 [SMILES: CC(C)COC(=O)CC1C(c2ccc(O)cc2)=NOC1c1ccccc1] | C21H24N2O3 | 353.41 |
| 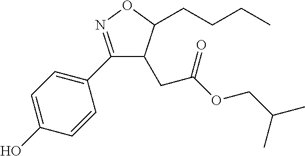 [SMILES: CCCCC1ON=C(c2ccc(O)cc2)C1CC(=O)OCC(C)C] | C19H28N2O3 | 333.42 |
| 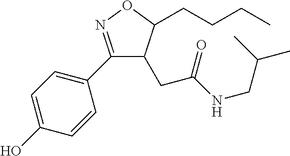 [SMILES: CCCCC1ON=C(c2ccc(O)cc2)C1CC(=O)NCC(C)C] | C19H28N2O3 | 332.44 |
| 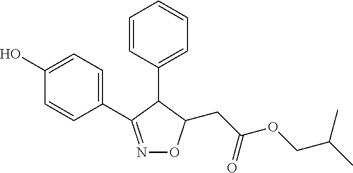 [SMILES: CC(C)COC(=O)CC1ON=C(c2ccc(O)cc2)C1c1ccccc1] | C21H23NO4 | 353.41 |
| 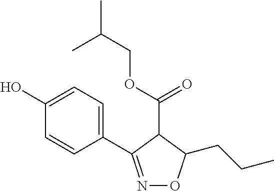 [SMILES: CCCC1ON=C(c2ccc(O)cc2)C1C(=O)OCC(C)C] | C17H23NO4 | 305.37 |
| 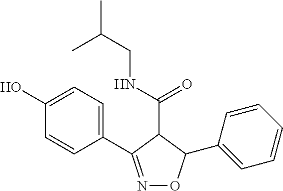 [SMILES: CC(C)CNC(=O)C1C(c2ccc(O)cc2)=NOC1c1ccccc1] | C20H22N2O3 | 338.40 |
| 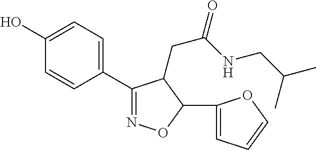 [SMILES: CC(C)CNC(=O)CC1C(c2ccc(O)cc2)=NOC1c1ccco1] | C19H22N2O4 | 342.39 |
| 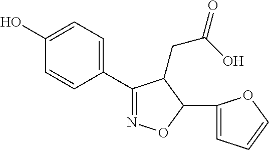 [SMILES: O=C(O)CC1C(c2ccc(O)cc2)=NOC1c1ccco1] | C15H13NO5 | 287.27 |
| 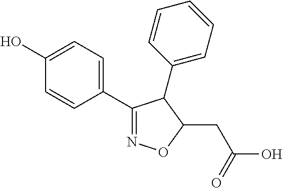 [SMILES: O=C(O)CC1ON=C(c2ccc(O)cc2)C1c1ccccc1] | C17H15NO4 | 297.31 |
| 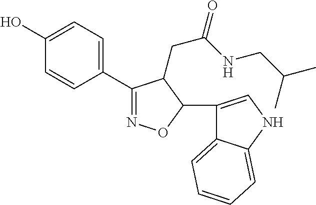 [SMILES: CC(C)CNC(=O)CC1C(c2ccc(O)cc2)=NOC1c1c[nH]c2ccccc12] | C23H25N3O3 | 391.46 |
| 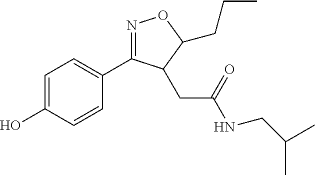 [SMILES: CCCC1ON=C(c2ccc(O)cc2)C1CC(=O)NCC(C)C] | C18H28N2O3 | 318.41 |
| 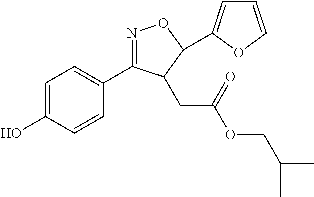 [SMILES: CC(C)COC(=O)CC1C(c2ccc(O)cc2)=NOC1c1ccco1] | C19H21NO5 | 343.37 |
| 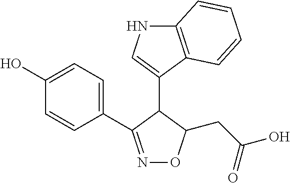 [SMILES: O=C(O)CC1ON=C(c2ccc(O)cc2)C1c1c[nH]c2ccccc12] | C19H16N2O4 | 336.34 |
| 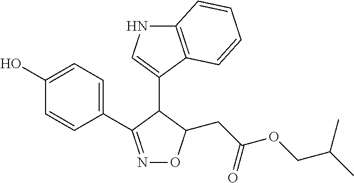 [SMILES: CC(C)COC(=O)CC1ON=C(c2ccc(O)cc2)C1c1c[nH]c2ccccc12] | C23H24N2O4 | 392.45 |
| 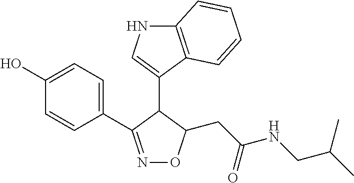 [SMILES: CC(C)CNC(=O)CC1ON=C(c2ccc(O)cc2)C1c1c[nH]c2ccccc12] | C23H25N3O3 | 391.46 |
| 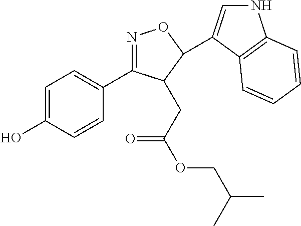 [SMILES: CC(C)COC(=O)CC1C(c2ccc(O)cc2)=NOC1c1c[nH]c2ccccc12] | C23H24N2O4 | 392.45 |
| 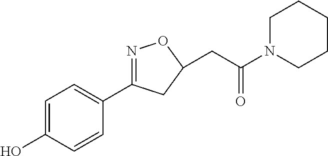 [SMILES: O=C(CC1CC(c2ccc(O)cc2)=NO1)N1CCCCC1] | C16H20N2O3 | 288.34 |
| 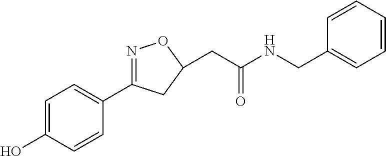 [SMILES: O=C(CC1CC(c2ccc(O)cc2)=NO1)NCc1ccccc1] | C18H18N2O3 | 310.35 |
| 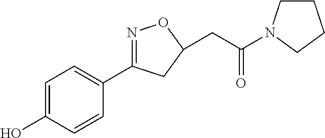 [SMILES: O=C(CC1CC(c2ccc(O)cc2)=NO1)N1CCCC1] | C15H18N2O3 | 274.32 |
| 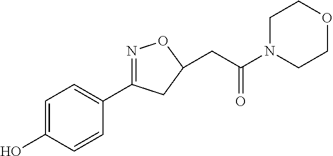 [SMILES: O=C(CC1CC(c2ccc(O)cc2)=NO1)N1CCOCC1] | C15H18N2O4 | 290.31 |
| 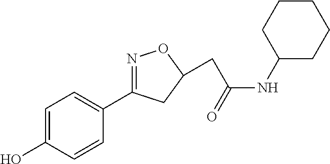 [SMILES: O=C(CC1CC(c2ccc(O)cc2)=NO1)NC1CCCCC1] | C17H22N2O3 | 302.37 |
| 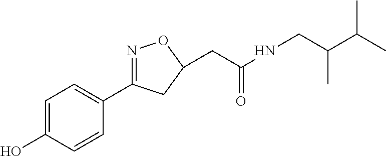 [SMILES: CC(C)C(C)CNC(=O)CC1CC(c2ccc(O)cc2)=NO1] | C17H24N2O3 | 304.38 |
| 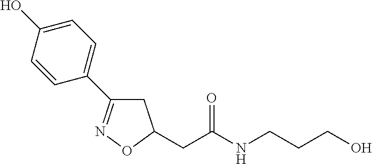 [SMILES: O=C(CC1CC(c2ccc(O)cc2)=NO1)NCCCO] | C14H18N2O4 | 278.3 |
| 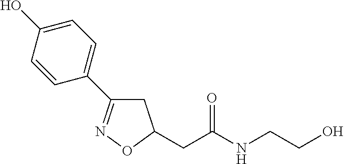 [SMILES: O=C(CC1CC(c2ccc(O)cc2)=NO1)NCCO] | C13H16N2O4 | 264.28 |
| 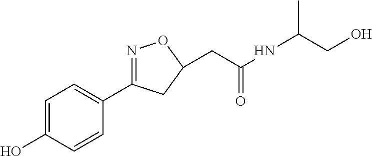 [SMILES: CC(CO)NC(=O)CC1CC(c2ccc(O)cc2)=NO1] | C14H18N2O4 | 278.3 |
| 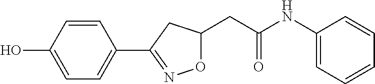 [SMILES: O=C(CC1CC(c2ccc(O)cc2)=NO1)Nc1ccccc1] | C17H16N2O3 | 296.32 |
| 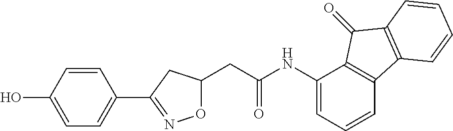 [SMILES: O=C(CC1CC(c2ccc(O)cc2)=NO1)Nc1cccc2c1C(=O)c1ccccc1-2] | C24H18N2O4 | 398.41 |
| 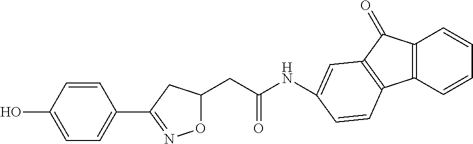 [SMILES: O=C(CC1CC(c2ccc(O)cc2)=NO1)Nc1ccc2c(c1)C(=O)c1ccccc1-2] | C24H18N2O4 | 398.41 |
| 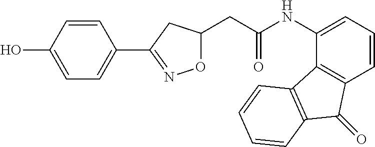 [SMILES: O=C(CC1CC(c2ccc(O)cc2)=NO1)Nc1cccc2c1-c1ccccc1C2=O] | C24H18N2O4 | 398.41 |
| 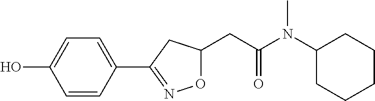 [SMILES: CN(C(=O)CC1CC(c2ccc(O)cc2)=NO1)C1CCCCC1] | C18H24N2O3 | 316.39 |
| 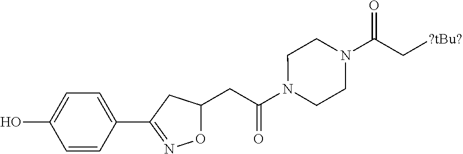 | C16H18N3O5 | 332.33 |
| 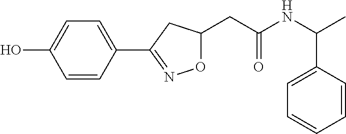 [SMILES: CC(NC(=O)CC1CC(c2ccc(O)cc2)=NO1)c1ccccc1] | C19H20N2O3 | 324.37 |
| 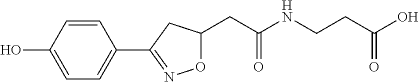 [SMILES: O=C(O)CCNC(=O)CC1CC(c2ccc(O)cc2)=NO1] | C14H16N2O5 | 292.29 |
| 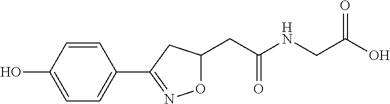 [SMILES: O=C(O)CNC(=O)CC1CC(c2ccc(O)cc2)=NO1] | C13H14N2O5 | 278.26 |
| 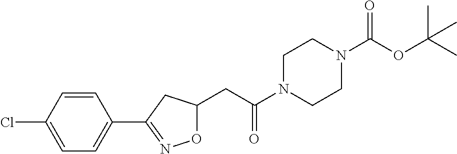 [SMILES: CC(C)(C)OC(=O)N1CCN(C(=O)CC2CC(c3ccc(Cl)cc3)=NO2)CC1] | C20H26N3O4Cl | 407.89 |
| 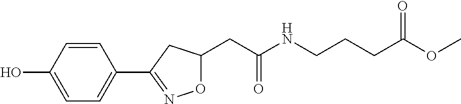 [SMILES: COC(=O)CCCNC(=O)CC1CC(c2ccc(O)cc2)=NO1] | C16H21N2O5 | 320.34 |
| 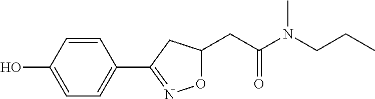 [SMILES: CCCN(C)C(=O)CC1CC(c2ccc(O)cc2)=NO1] | C15H21N2O3 | 276.35 |
| 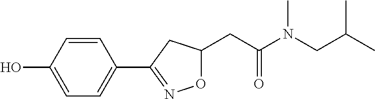 [SMILES: CC(C)CN(C)C(=O)CC1CC(c2ccc(O)cc2)=NO1] | C16H21N2O3 | 290.36 |
| 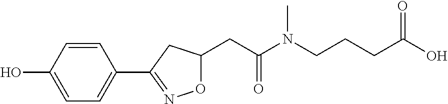 [SMILES: CN(CCCC(=O)O)C(=O)CC1CC(c2ccc(O)cc2)=NO1] | C16H20N2O5 | 320.34 |
| 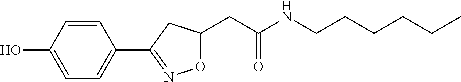 [SMILES: CCCCCCNC(=O)CC1CC(c2ccc(O)cc2)=NO1] | C18H26N2O3 | 304.38 |
| 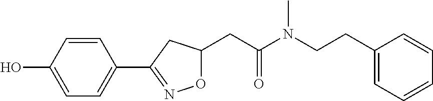 [SMILES: CN(CCc1ccccc1)C(=O)CC1CC(c2ccc(O)cc2)=NO1] | C20H22N2O3 | 338.4 |
| 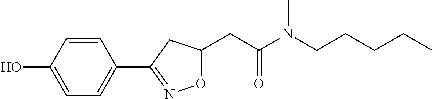 [SMILES: CCCCCN(C)C(=O)CC1CC(c2ccc(O)cc2)=NO1] | C17H24N2O3 | 304.38 |
| 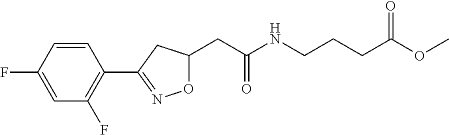 [SMILES: COC(=O)CCCNC(=O)CC1CC(c2ccc(F)cc2F)=NO1] | C16H18N2O4F2 | 340.32 |
| 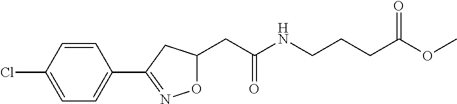 [SMILES: COC(=O)CCCNC(=O)CC1CC(c2ccc(Cl)cc2)=NO1] | C16H19N2O4Cl | 338.79 |
| 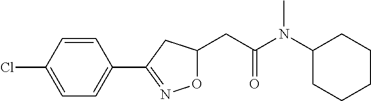 [SMILES: CN(C(=O)CC1CC(c2ccc(Cl)cc2)=NO1)C1CCCCC1] | C18H26N2O2Cl | 334.87 |
| 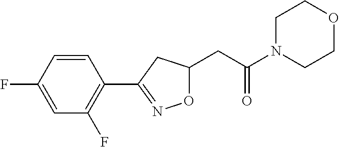 [SMILES: O=C(CC1CC(c2ccc(F)cc2F)=NO1)N1CCOCC1] | C15H16N2O3F2 | 310.28 |
| 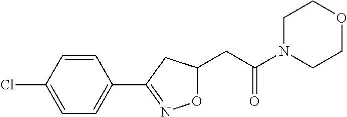 [SMILES: O=C(CC1CC(c2ccc(Cl)cc2)=NO1)N1CCOCC1] | C15H17N2O3Cl | 308.76 |
| 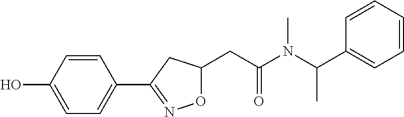 | C26H22N2O3 | 338.4 |
| 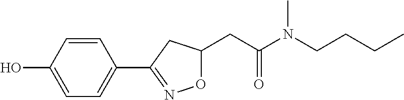 [SMILES: CCCCN(C)C(=O)CC1CC(c2ccc(O)cc2)=NO1] | C16H23N2O3 | 291.36 |
| 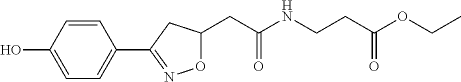 [SMILES: CCOC(=O)CCNC(=O)CC1CC(c2ccc(O)cc2)=NO1] | C16H19N2O5 | 320.34 |
| 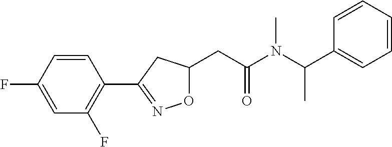 [SMILES: CC(c1ccccc1)N(C)C(=O)CC1CC(c2ccc(F)cc2F)=NO1] | C20H20N2O2F2 | 358.38 |
| 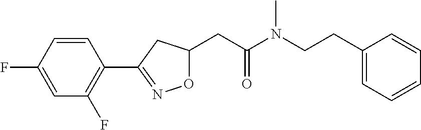 [SMILES: CN(CCc1ccccc1)C(=O)CC1CC(c2ccc(F)cc2F)=NO1] | C20H20N2O2F2 | 358.38 |
| 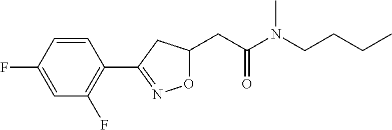 [SMILES: CCCCN(C)C(=O)CC1CC(c2ccc(F)cc2F)=NO1] | C16H20N2O2F2 | 310.34 |
| 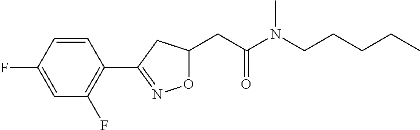 [SMILES: CCCCCN(C)C(=O)CC1CC(c2ccc(F)cc2F)=NO1] | C17H22N2O2F2 | 324.37 |
| 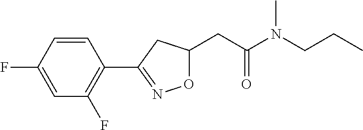 [SMILES: CCCN(C)C(=O)CC1CC(c2ccc(F)cc2F)=NO1] | C15H18N2O2F2 | 296.31 |
| 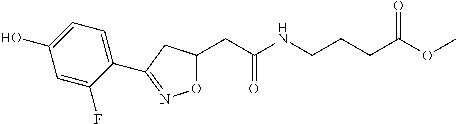 [SMILES: COC(=O)CCCNC(=O)CC1CC(c2ccc(O)cc2F)=NO1] | C19H18N2O5F | 338.33 |
| 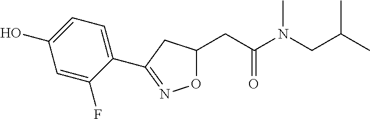 [SMILES: CC(C)CN(C)C(=O)CC1CC(c2ccc(O)cc2F)=NO1] | C16H21N2O3F | 308.35 |
| 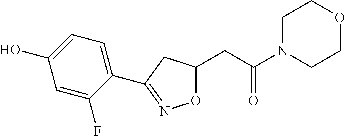 [SMILES: O=C(CC1CC(c2ccc(O)cc2F)=NO1)N1CCOCC1] | C15H17N2O4F | 308.3 |
| 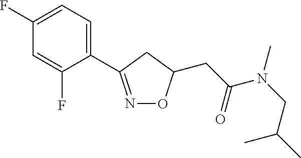 [SMILES: CC(C)CN(C)C(=O)CC1CC(c2ccc(F)cc2F)=NO1] | C16H20N2O2F2 | 310.33 |
| 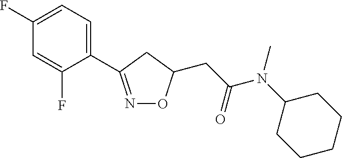 [SMILES: CN(C(=O)CC1CC(c2ccc(F)cc2F)=NO1)C1CCCCC1] | C18H22N2O2F2 | 336.37 |
| 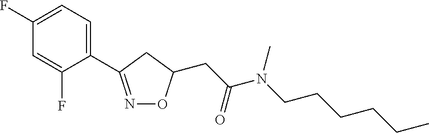 [SMILES: CCCCCCN(C)C(=O)CC1CC(c2ccc(F)cc2F)=NO1] | C18H24N2O2F2 | 338.39 |
| 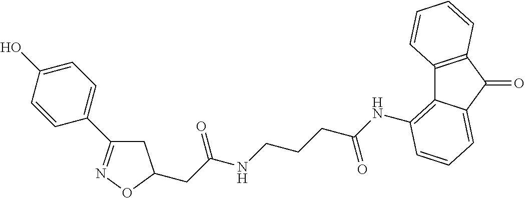 [SMILES: O=C(CC1CC(c2ccc(O)cc2)=NO1)NCCCC(=O)Nc1cccc2c1-c1ccccc1C2=O] | C28H25N3O5 | 483.5 |
| 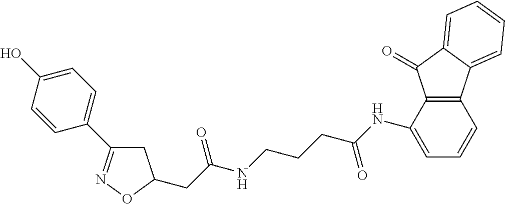 [SMILES: O=C(CC1CC(c2ccc(O)cc2)=NO1)NCCCC(=O)Nc1cccc2c1C(=O)c1ccccc1-2] | C28H25N3O5 | 483.5 |
| 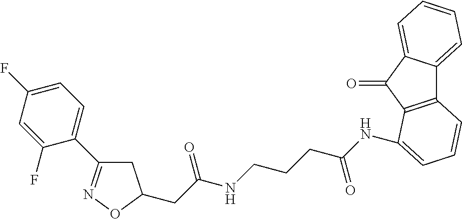 [SMILES: O=C(CC1CC(c2ccc(F)cc2F)=NO1)NCCCC(=O)Nc1cccc2c1C(=O)c1ccccc1-2] | C28H23N3O4F2 | 503.3 |
| 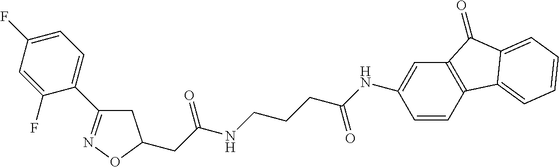 [SMILES: O=C(CC1CC(c2ccc(F)cc2F)=NO1)NCCCC(=O)Nc1ccc2c(c1)C(=O)c1ccccc1-2] | C28H23N3O4F2 | 503.3 |
| 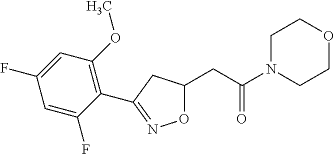 [SMILES: COc1cc(F)cc(F)c1C1=NOC(CC(=O)N2CCOCC2)C1] | C16H18N2O4F2 | 340.3 |
| 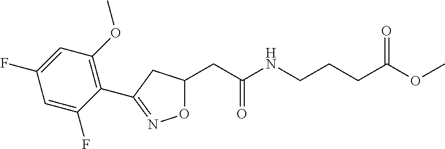 [SMILES: COC(=O)CCCNC(=O)CC1CC(c2c(F)cc(F)cc2OC)=NO1] | C17H20N2O5F2 | 370.35 |
| 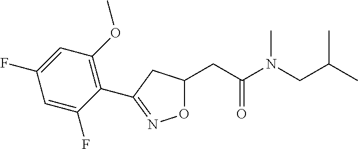 [SMILES: COc1cc(F)cc(F)c1C1=NOC(CC(=O)N(C)CC(C)C)C1] | C17H22N2O3F2 | 340.36 |
| 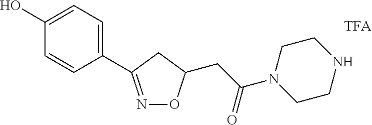 [SMILES: O=C(CC1CC(c2ccc(O)cc2)=NO1)N1CCNCC1] | C14H19O3N3•TFA | 403.35 |
| 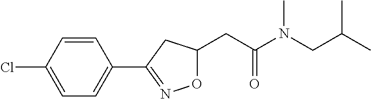 [SMILES: CC(C)CN(C)C(=O)CC1CC(c2ccc(Cl)cc2)=NO1] | C16H21N2O2Cl | 308.80 |
| 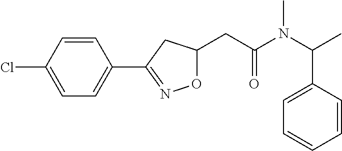 [SMILES: CC(c1ccccc1)N(C)C(=O)CC1CC(c2ccc(Cl)cc2)=NO1] | C20H21N2O2Cl | 356.85 |
| 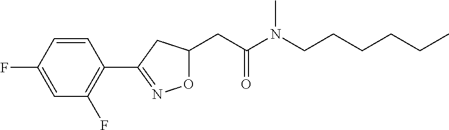 [SMILES: CCCCCCN(C)C(=O)CC1CC(c2ccc(F)cc2F)=NO1] | C18H24F2N2O2 | 338.39 |
| 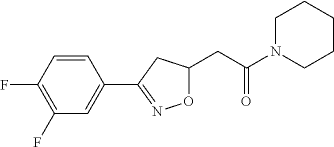 [SMILES: O=C(CC1CC(c2ccc(F)c(F)c2)=NO1)N1CCCCC1] | C16H18F2N2O2 | 308.32 |
| 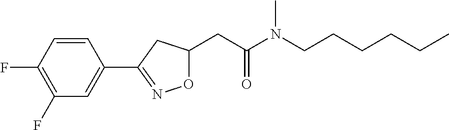 [SMILES: CCCCCCN(C)C(=O)CC1CC(c2ccc(F)c(F)c2)=NO1] | C18H24F2N2O2 | 338.39 |
| 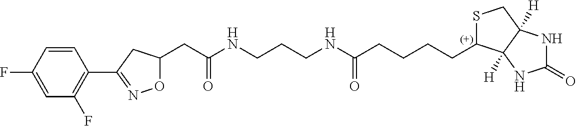 | C24H31F2N5O4S | 523.6 |
| 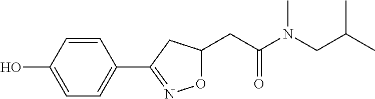 [SMILES: CC(C)CN(C)C(=O)CC1CC(c2ccc(O)cc2)=NO1] | C16H22N2O3 | 290.36 |
| 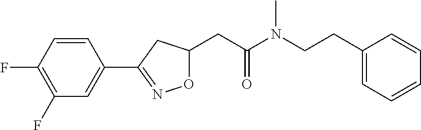 [SMILES: CN(CCc1ccccc1)C(=O)CC1CC(c2ccc(F)c(F)c2)=NO1] | C20H20F2N2O2 | 358.38 |
| 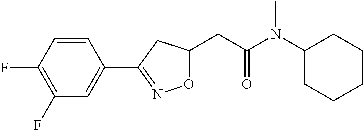 [SMILES: CN(C(=O)CC1CC(c2ccc(F)c(F)c2)=NO1)C1CCCCC1] | C18H22F2N2O2 | 336.38 |
| 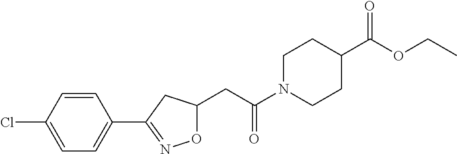 [SMILES: CCOC(=O)C1CCN(C(=O)CC2CC(c3ccc(Cl)cc3)=NO2)CC1] | C19H23ClN2O4 | 378.85 |
| 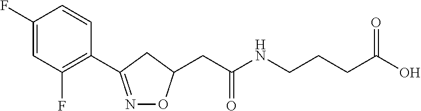 [SMILES: O=C(O)CCCNC(=O)CC1CC(c2ccc(F)cc2F)=NO1] | C15H16F2N2O4 | 326.30 |
| 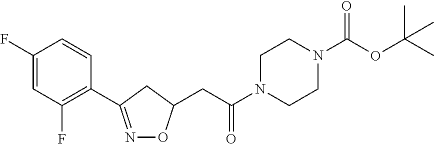 [SMILES: CC(C)(C)OC(=O)N1CCN(C(=O)CC2CC(c3ccc(F)cc3F)=NO2)CC1] | C20H25F2N3O4 | 409.4 |
| 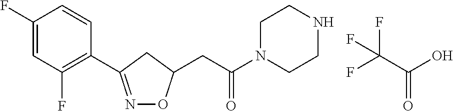 | C15H17F2N3O2•TFA | 423.33 |
| 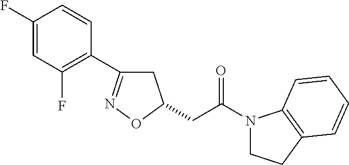 [SMILES: O=C(C[C@H]1CC(c2ccc(F)cc2F)=NO1)N1CCc2ccccc21] | C19H16F2N2O2 | 342.34 |
| 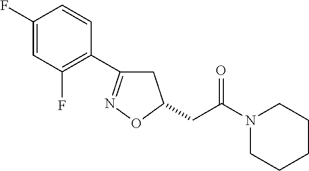 [SMILES: O=C(C[C@H]1CC(c2ccc(F)cc2F)=NO1)N1CCCCC1] | C16H18F2N2O2 | 308.32 |
| 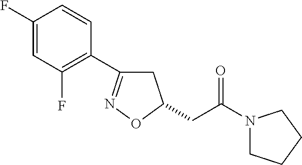 [SMILES: O=C(C[C@H]1CC(c2ccc(F)cc2F)=NO1)N1CCCC1] | C15H16F2N2O2 | 294.3 |
| 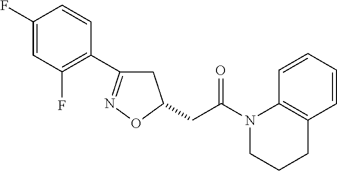 [SMILES: O=C(C[C@H]1CC(c2ccc(F)cc2F)=NO1)N1CCCc2ccccc21] | C20H18F2N2O2 | 356.37 |
| 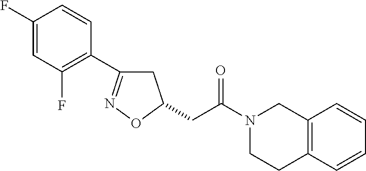 [SMILES: O=C(C[C@H]1CC(c2ccc(F)cc2F)=NO1)N1CCc2ccccc2C1] | C20H18F2N2O2 | 356.37 |
| 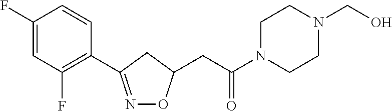 [SMILES: O=C(CC1CC(c2ccc(F)cc2F)=NO1)N1CCN(CO)CC1] | C16H19F2N3O3 | 339.34 |
| 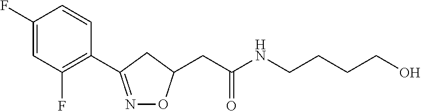 [SMILES: O=C(CC1CC(c2ccc(F)cc2F)=NO1)NCCCCO] | C15H18F2N2O3 | 312.31 |
| 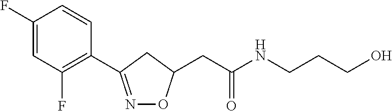 [SMILES: O=C(CC1CC(c2ccc(F)cc2F)=NO1)NCCCO] | C14H16F2N2O3 | 298.29 |
| 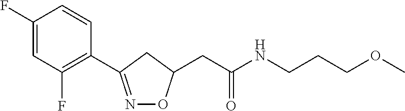 [SMILES: COCCCNC(=O)CC1CC(c2ccc(F)cc2F)=NO1] | C15H18F2N2O3 | 312.31 |
| 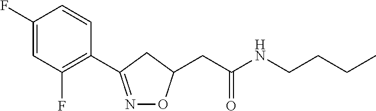 [SMILES: CCCCNC(=O)CC1CC(c2ccc(F)cc2F)=NO1] | C15H18F2N2O2 | 296.31 |
| 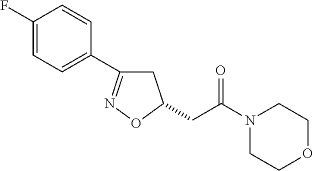 [SMILES: O=C(C[C@H]1CC(c2ccc(F)cc2)=NO1)N1CCOCC1] | C15H17FN2O3 | 292.31 |
| 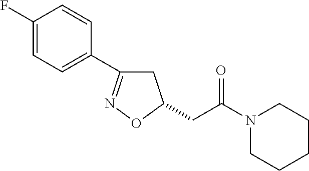 [SMILES: O=C(C[C@H]1CC(c2ccc(F)cc2)=NO1)N1CCCCC1] | C16H19FN2O2 | 290.33 |
| 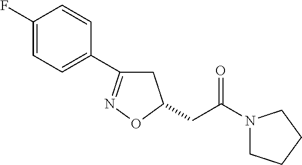 [SMILES: O=C(C[C@H]1CC(c2ccc(F)cc2)=NO1)N1CCCC1] | C15H17FN2O2 | 276.31 |
| 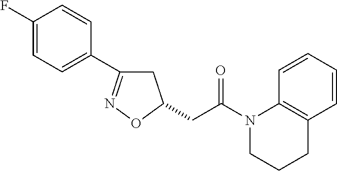 [SMILES: O=C(C[C@H]1CC(c2ccc(F)cc2)=NO1)N1CCCc2ccccc21] | C20H19FN2O2 | 338.38 |
| 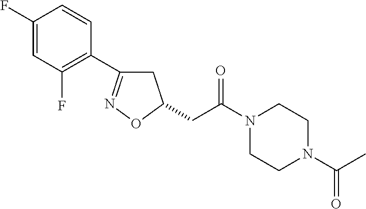 [SMILES: CC(=O)N1CCN(C(=O)C[C@H]2CC(c3ccc(F)cc3F)=NO2)CC1] | C17H19F2N3O3 | 351.35 |
| 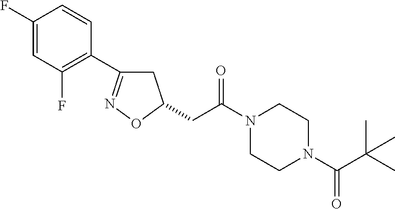 [SMILES: CC(C)(C)C(=O)N1CCN(C(=O)C[C@H]2CC(c3ccc(F)cc3F)=NO2)CC1] | C20H25F2N3O3 | 393.43 |
| 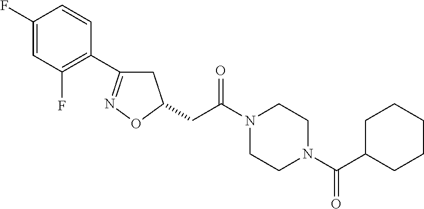 [SMILES: O=C(C[C@H]1CC(c2ccc(F)cc2F)=NO1)N1CCN(C(=O)C2CCCCC2)CC1] | C22H27F2N3O3 | 419.46 |
| 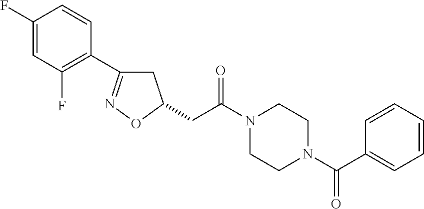 [SMILES: O=C(C[C@H]1CC(c2ccc(F)cc2F)=NO1)N1CCN(C(=O)c2ccccc2)CC1] | C22H21F2N3O3 | 413.42 |
| 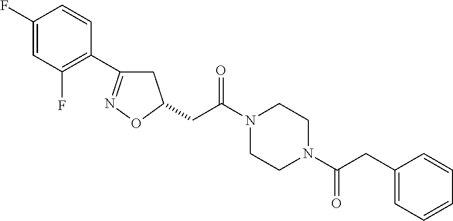 [SMILES: O=C(Cc1ccccc1)N1CCN(C(=O)C[C@H]2CC(c3ccc(F)cc3F)=NO2)CC1] | C23H23F2N3O3 | 427.44 |
| 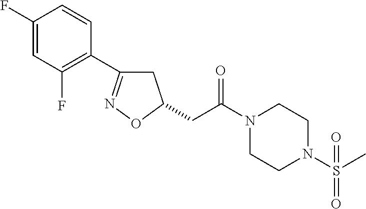 [SMILES: CS(=O)(=O)N1CCN(C(=O)C[C@H]2CC(c3ccc(F)cc3F)=NO2)CC1] | C16H19F2N3O4S | 387.40 |
| 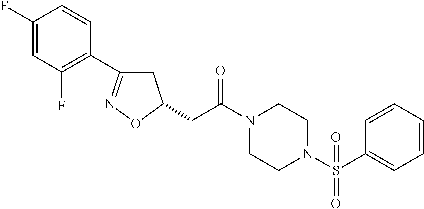 [SMILES: O=C(C[C@H]1CC(c2ccc(F)cc2F)=NO1)N1CCN(S(=O)(=O)c2ccccc2)CC1] | C21H21F2N3O4S | 449.47 |

Each of the following references is hereby incorporated by reference:
Cancer Facts and Figures. (American Cancer Society, 2008).
Fingerle-Rowson, G., et al. The p53-dependent effects of macrophage migration inhibitory factor revealed by gene targeting. Proc Natl Acad Sci USA 100, 9354-9359 (2003).
Petrenko, O. & Moll, U. M. Macrophage migration inhibitory factor MIF interferes with the Rb-E2F pathway. Mol Cell 17, 225-236 (2005).
Tomiyasu, M., Yoshino, I., Suemitsu, R., Okamoto, T. & Sugimachi, K. Quantification of macrophage migration inhibitory factor mRNA expression in non-small cell lung cancer tissues and its clinical significance. Clin Cancer Res 8, 3755-3760 (2002).
Bando, H., et al. Expression of macrophage migration inhibitory factor in human breast cancer: association with nodal spread. Jpn J Cancer Res 93, 389-396 (2002).
Meyer-Siegler, K. & Hudson, P. B. Enhanced expression of macrophage migration inhibitory factor in prostatic adenocarcinoma metastases. Urology 48, 448-452 (1996).
David, J. R. Delayed hypersensitivity in vitro: its mediation by cell-free substances formed by lymphoid cell-antigen interaction. Proc Natl Acad Sci USA 56, 72-77 (1966).
Calandra, T. & Bucala, R. Macrophage migration inhibitory factor (MIF): a glucocorticoid counter-regulator within the immune system. Crit Rev Immunol 17, 77-88 (1997).
Hudson, J. D., et al. A proinflammatory cytokine inhibits p53 tumor suppressor activity. J Exp Med 190, 1375-1382 (1999).
Petrenko, O., Fingerle-Rowson, G., Peng, T., Mitchell, R. A. & Metz, C. N. Macrophage migration inhibitory factor deficiency is associated with altered cell growth and reduced susceptibility to Ras-mediated transformation. J Biol Chem 278, 11078-11085 (2003).
Lue, H., et al. Macrophage migration inhibitory factor (MIF) promotes cell survival by activation of the Akt pathway and role for CSN5/JAB1 in the control of autocrine MIF activity. Oncogene (2007).
Ren, Y., et al. Inhibition of tumor growth and metastasis in vitro and in vivo by targeting macrophage migration inhibitory factor in human neuroblastoma. Oncogene (2006).
Hagemann, T., et al. Macrophages induce invasiveness of epithelial cancer cells via NF-kappaB and JNK. J Immunol 175, 1197-1205 (2005).
Meyer-Siegler, K. L. & Vera, P. L. Substance P induced release of macrophage migration inhibitory factor from rat bladder epithelium. J Urol 171, 1698-1703 (2004).
Meyer-Siegler, K. L., Leifheit, E. C. & Vera, P. L. Inhibition of macrophage migration inhibitory factor decreases proliferation and cytokine expression in bladder cancer cells. BMC Cancer 4, 34 (2004).
Sun, B., et al. Induction of macrophage migration inhibitory factor by lysophosphatidic acid: relevance to tumor growth and angiogenesis. Int J Mol Med 12, 633-641 (2003).
Meyer-Siegler, K. L., Iczkowski, K. A., Leng, L., Bucala, R. & Vera, P. L. Inhibition of macrophage migration inhibitory factor or its receptor (CD74) attenuates growth and invasion of DU-145 prostate cancer cells. J Immunol 177, 8730-8739 (2006).
Taylor, J. A., 3rd, et al. Null mutation for macrophage migration inhibitory factor (MIF) is associated with less aggressive bladder cancer in mice. BMC Cancer 7, 135 (2007).
Sylvester, R. J., van der, M. A. & Lamm, D. L. Intravesical bacillus Calmette-Guerin reduces the risk of progression in patients with superficial bladder cancer: a meta-analysis of the published results of randomized clinical trials. J Urol 168, 1964-1970 (2002).

### EXAMPLES

The claimed subject matter may be described in further detail with reference to the following examples. The examples are intended to be illustrative, but the claimed subject matter is not considered as being limited to the materials, conditions, or process parameters set forth in the examples. All parts and percentages are by unit weight unless otherwise indicated.

The present examples describe the impact of two compounds, CPSI-2705 and CPSI-1306, (Cytokine PharmaSciences, Inc, King of Prussia, Pa.; CPSI) administered orally in the BBN model of bladder cancer. The BBN model is an accepted in vivo model for bladder cancer.

Materials & Methods:

The exemplary compounds are shown below.

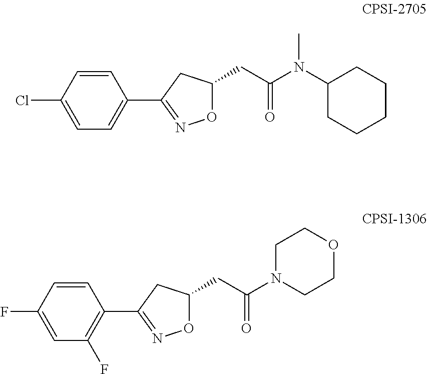 [SMILES: CN(C(=O)C[C@H]1CC(c2ccc(Cl)cc2)=NO1)C1CCCCC1 ; O=C(C[C@H]1CC(c2ccc(F)cc2F)=NO1)N1CCOCC1]

Both CPSI-2705 and 1306 were used in the R configurations.

Three-month old C57Bl/6 mice were purchased from Jackson Laboratories (Bar Harbor, Me.) and Charles River (Wilmington, Mass.). The animals were maintained at the University of Connecticut Center for Laboratory Animal Care under National Institutes of Health guidelines. All procedures were approved by an institutional animal care committee. The mice were housed in a controlled environment with a 12 hour light-12 hour dark cycle and provided food and water ad libitum.

All animals received BBN(N-butyl-N-(4-hydroxybutyl)-nitrosamine, TCI America, Portland, Oreg.) 0.05% in water in brown bottles for 22 weeks. BBN is known to induce cancer in the bladder. Mice were treated with either the CPSI compounds or vehicle for weeks 16-22. Timing of dosing was intended to correspond with the timeframe of tumor progression from CIS to invasive disease, as determined in prior experiments.

Experiment 1:

Thirty 3-month old male mice (n=10/group) were gavage fed either vehicle (PEG 300, methylcellulose) (control group), CPSI-2705, or CPSI-1306 at 25 mg/kg for weeks 16-22.

Experiment 2:

Sixty 3-month old male mice (n=˜8/group) were gavage fed an escalating dose of CPSI-1306 (0.001, 0.01, 0.1, 1.0, 10 or 25 mg/kg) or vehicle (PEG 400, H₂O) for weeks 16-22.

Animals were inspected daily for general health with weights recorded at least biweekly and more frequently in the last several weeks based on recorded weight loss. Animals were euthanized by CO₂ inhalation and cervical dislocation. Bladders were harvested, weighed, placed in PBS/formaldehyde for 24 hours and then transferred to PBS. Upper tracts (kidney and ureters) were inspected for hydronephrosis. Bladders were assessed for tumor stage and grade and amount of pathologic involvement with invasive disease. Bladders were sectioned in the mid-sagital plane, laid flat on the medial surface and step sectioned for whole mount pathologic analysis. Haematoxylin and eosin (H&E) staining was carried out to assess for histology and pathology. Bladders were step sectioned, embedded in paraffin, serially sectioned and stained with H&E and assessed for tumor stage and grade and amount of pathologic involvement with invasive disease. A single pathologist reviewed all slides in a blinded manner to provide a pathological diagnosis. As a tumor develops in the bladder, the size and weight of the bladder increases. Bladder weight is therefore a useful surrogate for bladder tumor burden, representing the relative amount of bladder tumor that develops during continual exposure to the carcinogen, BBN.

Immunohistochemistry (IHC) for platelet/endothelial cell adhesion molecule 1 (PECAM-1) was performed for angiogenesis. Endogenous peroxidase activity was blocked with 3% H2O2. Non-specific staining was blocked with Power Block™. Sections were stained with a goat polyclonal anti-PECAM antibody (Santa Cruz Biotechnology, M20 SC-1506) at 1:500 dilution in PBS-BSA. Bound anti-PECAM-1 antibody was detected with a secondary biotinylated horse-anti-goat antibody (1:200) and visualized using DAB. Microvessel density was calculated as the mean # vessels from 3 determined “hot spots” at the tumor-stromal interface per specimen and the subsequent mean for the group.

Values shown are means±SEM. Data that did not pass the Shapiro-Wilk test for normality were log transformed. Statistical analysis was performed using SPSS® version 2.03 (San Rafael, Calif.). Differences were examined by one-way analysis of variance (ANOVA) followed by the post-hoc Dunnett's test.

Results:

All groups showed equal weight gain through week 16 and appeared to be in generally good health.

Experiment 1:

There was a 16% weight loss from the start of gavage treatment in the control group compared to 10-11% in the treated groups. In general, the group receiving CPSI-1306 appeared healthier during the time of drug treatment compared to the other two treatment arms. Two animals each from the control and CPSI-2705 group died prior to completion of the study. Their bladders were harvested within 12 hours of death and included in the final analysis. At euthanasia the majority of control animals had evidence of upper tract obstruction with severe hydro-ureteronephrosis. Fewer animals were noted to have obstruction in the CPSI-2705 group and none seen in the CPSI-1306 group with essentially normal upper tracts. Average bladder weights for the groups were 0.5±0.5 gm, 0.3±0.3 gm and 0.2±0.2 gm for control, CPSI-2705 and CPSI-1306 respectively which approached statistical significance for control vs CPSI-1306 (log transformed, t-test, p=0.06). Pathologically there was a higher proportion of T3 disease in the control (80%) vs treated groups (60%).

Tumor burden was markedly diminished in the treatment groups (Table 1) with a mean of 81% (SD±20%) of the tissue noted to be involved with invasive disease in the control vs 62% (SD±30%) and 59% (SD±32%) involvement in CPSI-2705 and CPSI-1306 groups respectively (p=0.07 for control vs CPSI-1306). Several bladders in the treatment arms were noted to have only focal areas of invasion. Tumor grade trended lower in the treatment groups as well with more metaplasia rather than dysplasia noted.

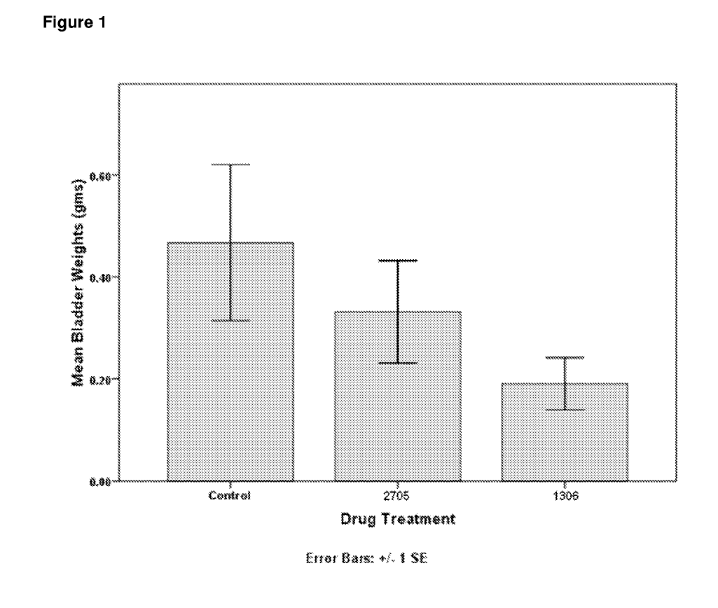

FIG. 1 shows the mean bladder weight for each experimental group in Experiment 1: the control group of animals, the animals receiving the CPSI-2705 compound, and the animals receiving the CPSI-1306 compound. The mean bladder weight for those animals receiving the CPSI-1306 compound was 2.5 fold less than that of the control group (p=0.06). The bars shown in the graph represent one standard error of the mean.

TABLE 1
Pathologic results for groups.

| | | | Pieces | | | Squamous | |
|---|---|---|---|---|---|---|---|
| Num- | | Pieces/ | involved | | | met and | Keratin- |
| ber | Group | sections | (inv + in situ %) | | Stage | normal | ization |
| 1 | C | 8 | 8 | (90 + 5%) | T3 | <5% | +++ |
| 2 | C | 4 | 4 | (90 + 5%) | T3 | <5% | +++ |
| 3 | C | 4 | 4 | (90 + 5%) | T3 | <5% | +++ |
| 4 | C | 4 | 4 | (90 + 5%) | T2 | <5% | +++ |
| 5 | C | 2 | 2 | (70 + 10%) | T1 | 20% | +++ |
| 6 | C | 3 and 1 | 3 | (90 + 5%) | T3 | 10% | ++ |
| | | ureter only | (ureter neg) | | | | |
| 7 | C | >8 | >8 | (90 + 5%) | T3 | <5% | +++ |
| 8 | C | 7 | 7 | (75 + 5%) | T3 | 20% | +++ |
| 27 | C | 7 | 7 | (30%) | T3 | 70% | + |
| 28 | C | 4 | 4 | (95%) | T3 | <5% | − |
| 9 | 2705 | 8 | 8 | (80 + 10%) | T3 | 10% | +++ |
| 10 | 2705 | 8 | 8 | (85 + 5%) | T3 | 10% | +++ |
| 11 | 2705 | 2 | 2 | (60%) | T2 | 40% | − |
| 12 | 2705 | 6 | 6 | (70%) | T3 | 30% | +++ |
| 13 | 2705 | 4 | 4 | (60 + 10%) | T3 | 30% | + |
| 14 | 2705 | 2 | 2 | (30%) | T1 | 70% | + |
| 15 | 2705 | 4 | 1 − inv, 3 − in situ | | T1 | 70% | + |
| | | | (10 + 20%) | | | | |
| 16 | 2705 | 4 | 4 | (30 + 5%) | T1 | 65% | ++ |
| 29 | 2705 | 10 | 10 | (95%) | T3 | <5% | +++ |
| 30 | 2705 | 8 | 8 | (95%) | T3 | <5% | +++ |
| 17 | 1306 | 4 | 4 | (25 + 5%) | T2 | 70% | ++ |
| | | | | | | with focal | |
| | | | | | | dysplasia | |
| 18 | 1306 | 4 | 4 | (15 + 15%) | T2 | 70% | +++ |
| 19 | 1306 | 2 | 2 | (20%) | T1 | 80% | − |
| | | | | | | with focal | |
| | | | | | | dysplasia | |
| 20 | 1306 | 4 | 4 | (90%) | T3 | 10% | − |
| 21 | 1306 | 7 | 7 | (95%) | T3 | <5% | ++ |
| 22 | 1306 | 4 | 4 | (35 + 10%) | T3 | 55% | + |
| | | | | | | with focal | |
| | | | | | | dysplasia | |
| 23 | 1306 | 6 | 6 | (90%) | T3 | 10% | +++ |
| | | | | | | with focal | |
| | | | | | | dysplasia | |
| 24 | 1306 | 4 | 3 − inv, 1 − in situ | | T1 | 25% | − |
| | | | (70 + 5%) | | | | |
| 25 | 1306 | 4 | 4 | (80 + 10%) | T3 | 10% | ++ |
| 26 | 1306 | 4 | 4 | (65 + 15%) | T3 | 20% | ++ |

C = control, 2705 = CPSI-2705, 1306 = CPSI-1306.

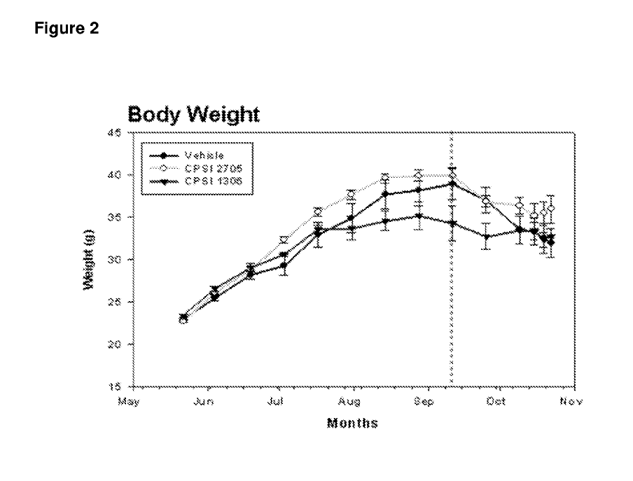

FIG. 2 shows mean body weights from Experiment 1. Dashed line indicates initiation of treatment with oral inhibitors. Treated groups had a lower % weight loss during the drug treatment time compared to vehicle treated mice.

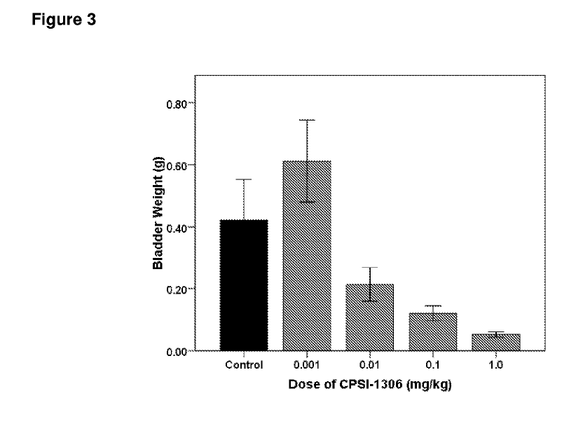

FIG. 3 shows mean bladder weights from Experiment 2 at euthanasia as surrogate for tumor volume for dose escalation study of CPSI-1306 given during weeks 16-22. Animals lost due to fighting prior to drug treatment were removed from the 2 highest doses (25 & 10 mg/kg) resulting in group numbers too small to perform analysis. (*, p<0.05 for 0.1 mg/kg and 1.0 mg/kg vs vehicle for Dunnett post-hoc, ANOVA p=0.004). Bars represent +/−1 SE.

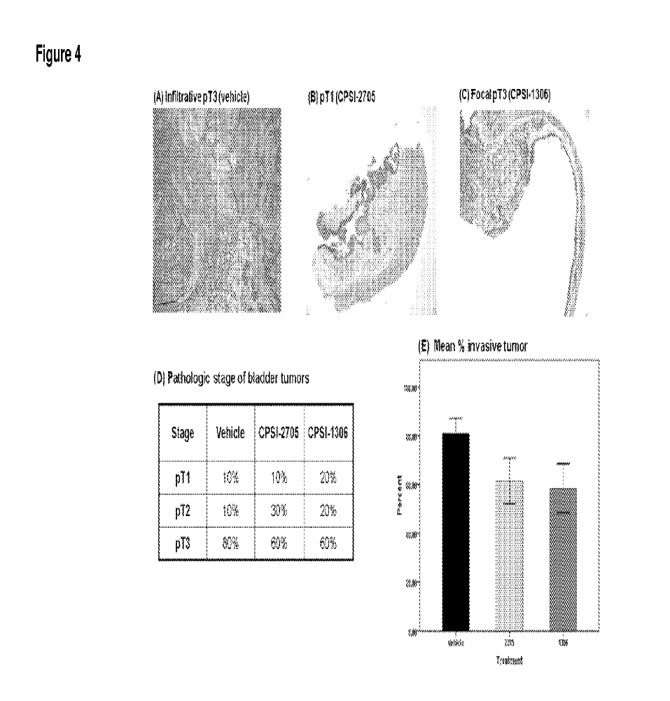

FIG. 4 shows bladder pathology for Experiment 1 in mice fed vehicle or CPSI oral inhibitor 2705 or 1306 at 25 mg/kg for weeks 16-22. H&E slides of pathology representing: infiltrative pT3 cancer in the vehicle-treated group (FIG. 4A); pT1 disease in the CPSI-2705 group (FIG. 4B); and focal pT3 with areas of normal mucosa in the CPSI-1306 group (FIG. 4C). FIG. 4D shows percentage of bladders in Experiment 1 treatment groups with each pathologic stage. A decrease in the percentage of pT3 tumors was seen in the treated groups. FIG. 4 E shows mean percentage of tumors representing invasive disease in each group for Experiment 1. Smaller tumors with a smaller percentage of invasiveness was seen in the CPSI-2705 and CPSI-1306 treated groups from Experiment 1. Bars are means+/−SE.

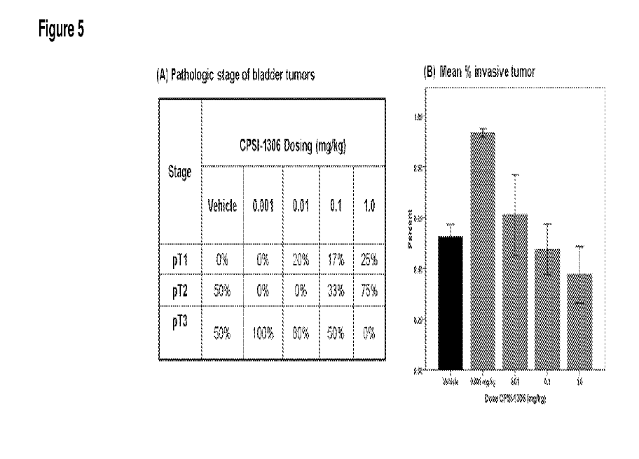

FIG. 5 shows bladder pathology from Experiment 2. FIG. 5A shows percentage of bladders in each dosing group with each pathologic stage. A decrease in the percentage of higher stage tumors was seen with drug dosing with more pT1 tumors than seen in the vehicle and lower dose groups. FIG. 5B shows mean percentage of tumor representing invasive disease in each group. Smaller tumors with a smaller percentage of invasiveness was seen in the CPSI-1306 dosing groups. Bars are means+/−1 SE.

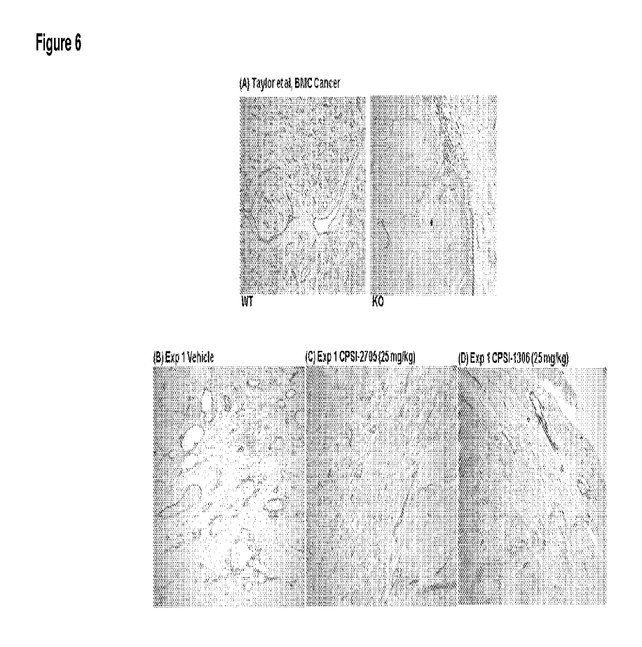

FIG. 6 shows tumor associated angiogenesis (via PECAM-1 IHC staining for vascular structures) from a previously published gene deletion model (FIG. 6A) and from Experiment 1 (FIGS. 6B, C, and D). FIG. 6A shows previously published results in WT vs KO animals in the BBN model. Microvessel density analysis revealed a statistically significant difference (Taylor et al, BMC Cancer. 2007, 7:135). FIGS. 6B, C, and D present data from experiment 1 in animals receiving vehicle (FIG. 6B); CPSI-2705 (FIG. 6C); and CPSI-1306 (FIG. 6D). A trend was noted across treatment groups for lower tumor associated stromal angiogenesis. Formal MVD analyses did not reach statistical significance.

In the present studies, mice were first treated orally with either vehicle or one of two inhibitors of MIF (Experiment 1). Then, a dose response study was carried out using the most efficacious compound identified in the first experiment (Experiment 2). In both studies, the oral inhibitors were associated with a reduction in tumor burden and percentage of invasive tumor. There was a trend toward lower stage as well as tumor-stromal interface angiogenesis. In the dose-response study, a significant effect was seen for treatment with CPSI-1306 at 1.0 mg/kg and 0.1 mg/kg compared to vehicle. These are the first studies to demonstrate a significant effect of oral MIF inhibitors on tumor development in a model of bladder cancer.

It is unexpected and surprising that oral administration of MIF inhibitors resulted in decreased weight loss associated with bladder cancer, improved general health, decreased bladder cancer tumor burden by bladder weight and pathologic assessment, and a trend toward both lower stage of bladder cancer and lower grade of bladder cancer. Absent any evidence to the contrary, the present inventors believe that the results observed for the two exemplary compounds would be the same or similar to that for the other compounds described herein.

This application is based on and claims priority to U.S. Provisional Application Ser. No. 61/296,182, filed Jan. 19, 2010, the entire contents of which are hereby incorporated by reference.

1. A method, comprising treating or preventing at least one disease selected from the group consisting of bladder cancer, weight loss accompanying bladder cancer, bone pain accompanying bladder cancer, bladder cancer carcinoma in-situ (CIS), TA stage bladder cancer, T1 stage bladder cancer, T2 stage bladder cancer, T3 stage bladder cancer, T4 stage bladder cancer, lymph node metastasis therefrom, solid organ metastasis therefrom, bony metastasis therefrom, cachexia/anorexia associated with bladder cancer, generalized wasting syndrome associated with bladder cancer, or a combination thereof, by administering to a subject in need thereof one or more compounds having the following formula (I), prodrug thereof, salt thereof, or a combination thereof, optionally in contact with a pharmaceutically acceptable carrier or excipient: 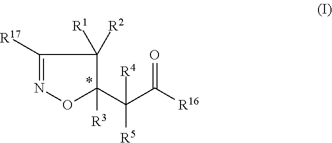
wherein at least the carbon marked “*” is chiral;
wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;
wherein R¹⁶ is hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms
and wherein R¹⁷ is an alkyl group, a cycloalkyl group, an alkenyl group, an alkynyl group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

2. The method according to claim 1, wherein the compound having formula (I) has the following formula (II), prodrug thereof, salt thereof, or a combination thereof: 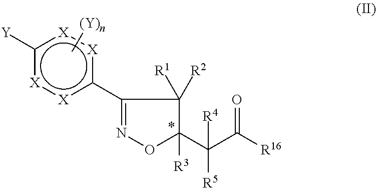
wherein * denotes a chiral carbon;
wherein each X is independently carbon or nitrogen, wherein when any X is carbon, it comprises a Y substituent, n being an integer of from 1 to 4 and being the number of X's that are carbon;
and wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms.

3. The method according to claim 2, wherein the structure: 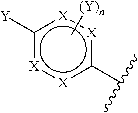
is one of the following three structures:

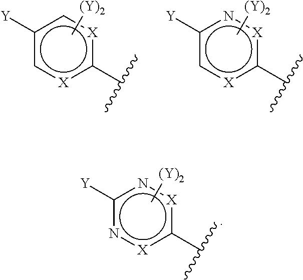

4. The method according to claim 2, wherein the structure 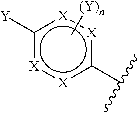
is one of the following structures:

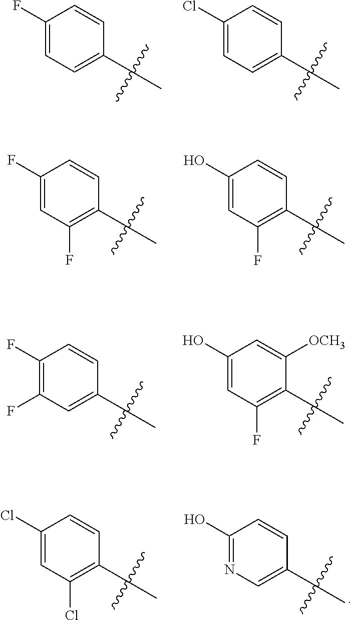

5. The method according to claim 1, wherein R¹⁶ is one of the following structures:

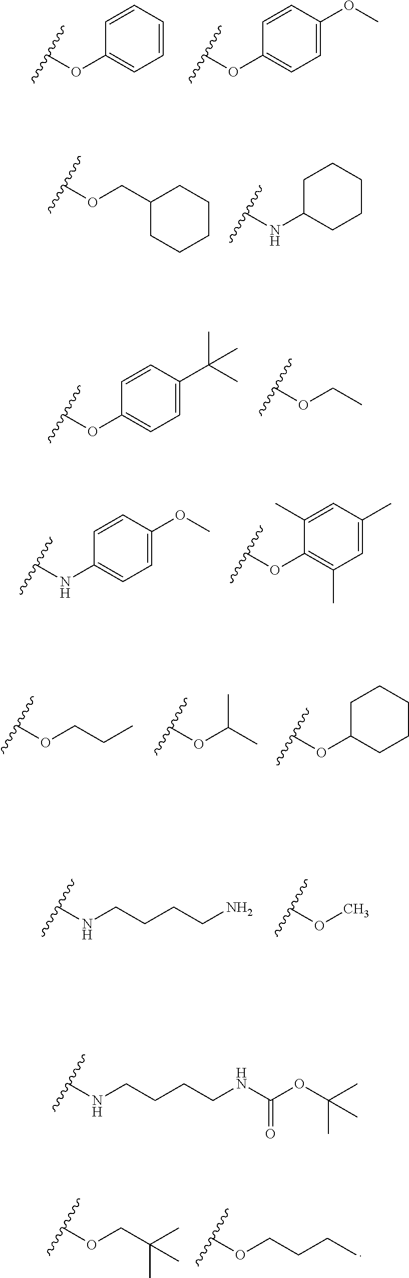

6. A package, comprising:
the compound having the following formula (I), prodrug thereof, salt thereof, or a combination thereof, optionally in contact with a pharmaceutically acceptable carrier or excipient; and
instructions for treating or preventing at least one disease selected from the group consisting of bladder cancer, weight loss accompanying bladder cancer, bone pain accompanying bladder cancer, bladder cancer carcinoma in-situ (CIS), TA stage bladder cancer, T1 stage bladder cancer, T2 stage bladder cancer, T3 stage bladder cancer, T4 stage bladder cancer, lymph node metastasis therefrom, solid organ metastasis therefrom, bony metastasis therefrom, cachexia/anorexia associated with bladder cancer, generalized wasting syndrome associated with bladder cancer, or a combination thereof, by administering to a subject in need thereof one or more compounds having the formula (I), prodrug thereof, salt thereof, or a combination thereof, optionally in contact with a pharmaceutically acceptable carrier or excipient: 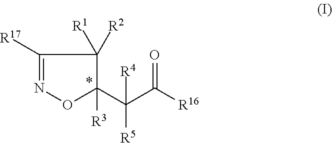
wherein at least the carbon marked “*” is chiral;
wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;
wherein R¹⁶ is hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms
and wherein R¹⁷ is an alkyl group, a cycloalkyl group, an alkenyl group, an alkynyl group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

7. The package according to claim 6, wherein the compound having formula (I) has the following formula (II), prodrug thereof, salt thereof, or a combination thereof: 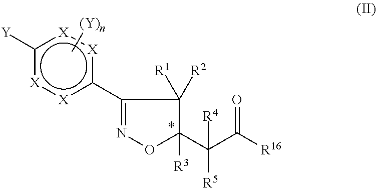
wherein * denotes a chiral carbon;
wherein each X is independently carbon or nitrogen, wherein when any X is carbon, it comprises a Y substituent, n being an integer of from 1 to 4 and being the number of X's that are carbon;
and wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms.

8. The package according to claim 7, wherein the structure: 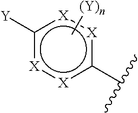
is one of the following three structures:

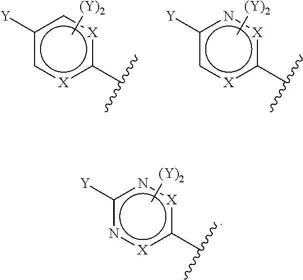

9. The package according to claim 7, wherein the structure 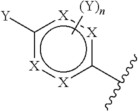
is one of the following structures:

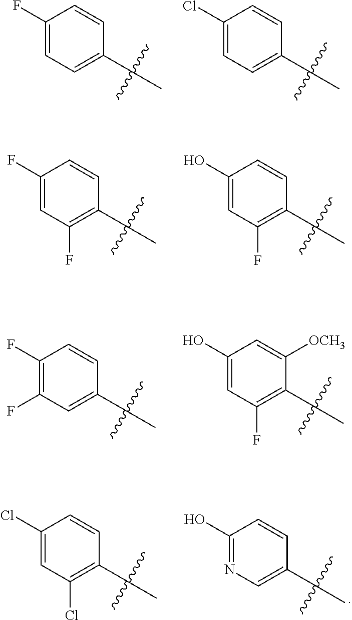

10. The package according to claim 6, wherein R¹⁶ is one of the following structures:

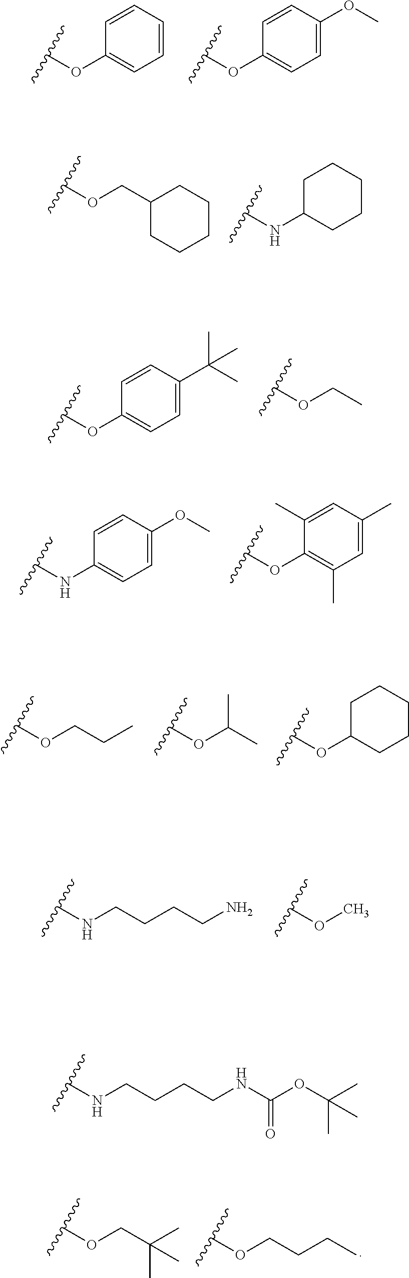

11. A use of one or more compounds having the following formula (I), prodrug thereof, salt thereof, or a combination thereof: 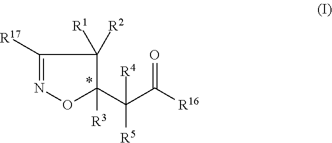
for treating or preventing at least one disease selected from the group consisting of bladder cancer, weight loss accompanying bladder cancer, bone pain accompanying bladder cancer, bladder cancer carcinoma in-situ (CIS), TA stage bladder cancer, T1 stage bladder cancer, T2 stage bladder cancer, T3 stage bladder cancer, T4 stage bladder cancer, lymph node metastasis therefrom, solid organ metastasis therefrom, bony metastasis therefrom, cachexia/anorexia associated with bladder cancer, generalized wasting syndrome associated with bladder cancer, or a combination thereof,
wherein at least the carbon marked “*” is chiral;
wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;
wherein R¹⁶ is hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms
and wherein R¹⁷ is an alkyl group, a cycloalkyl group, an alkenyl group, an alkynyl group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

12. The use according to claim 11, wherein the compound having formula (I) has the following formula (II), prodrug thereof, salt thereof, or a combination thereof: 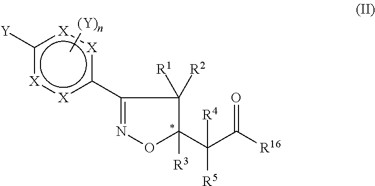
wherein * denotes a chiral carbon;
wherein each X is independently carbon or nitrogen, wherein when any X is carbon, it comprises a Y substituent, n being an integer of from 1 to 4 and being the number of X's that are carbon;
and wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms.

13. The use according to claim 12, wherein the structure: 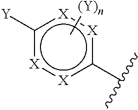
is one of the following three structures:

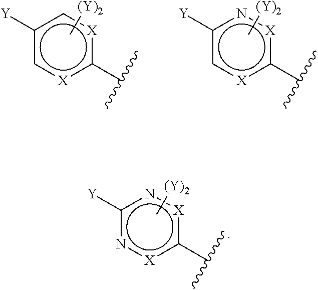

14. The use according to claim 12, wherein the structure 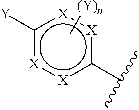
is one of the following structures:

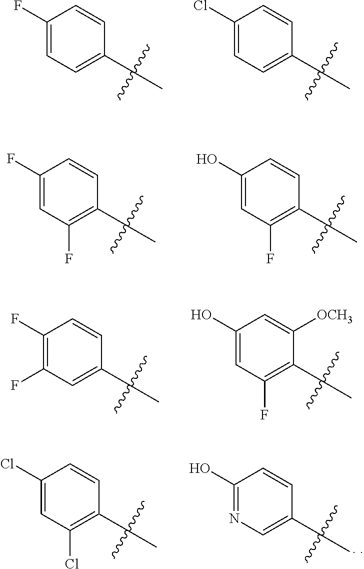

15. The use according to claim 11, wherein R¹⁶ is one of the following structures:

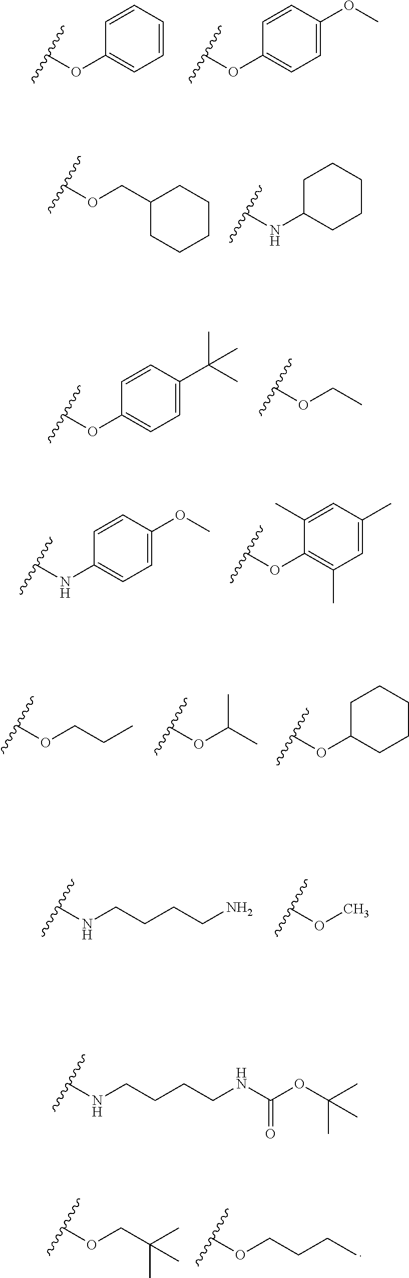

16. A use of one or more compounds having the following formula (I), prodrug thereof, salt thereof, or a combination thereof: 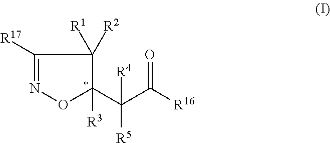
in the preparation of a medicament for treating or preventing at least one disease selected from the group consisting of bladder cancer, weight loss accompanying bladder cancer, bone pain accompanying bladder cancer, bladder cancer carcinoma in-situ (CIS), TA stage bladder cancer, T1 stage bladder cancer, T2 stage bladder cancer, T3 stage bladder cancer, T4 stage bladder cancer, lymph node metastasis therefrom, solid organ metastasis therefrom, bony metastasis therefrom, cachexia/anorexia associated with bladder cancer, generalized wasting syndrome associated with bladder cancer, or a combination thereof,
wherein at least the carbon marked “*” is chiral;
wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;
wherein R¹⁶ is hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms
and wherein R¹⁷ is an alkyl group, a cycloalkyl group, an alkenyl group, an alkynyl group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

17. The use according to claim 16, wherein the compound having formula (I) has the following formula (II), prodrug thereof, salt thereof, or a combination thereof: 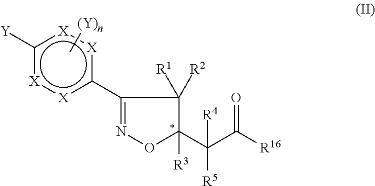
wherein * denotes a chiral carbon;
wherein each X is independently carbon or nitrogen, wherein when any X is carbon, it comprises a Y substituent, n being an integer of from 1 to 4 and being the number of X's that are carbon;
and wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms.

18. The use according to claim 17, wherein the structure: 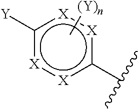
is one of the following three structures:

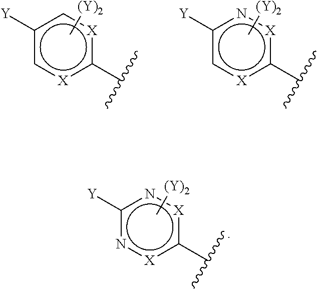

19. The use according to claim 17, wherein the structure 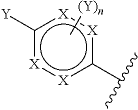
is one of the following structures:

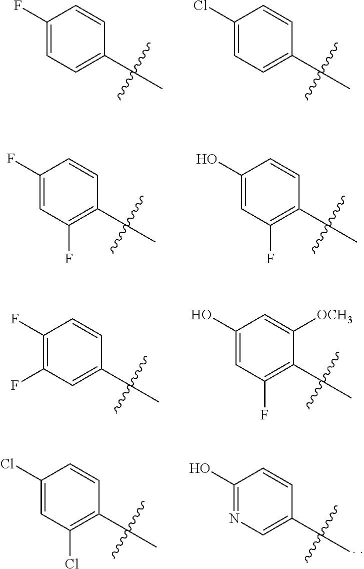

20. The use according to claim 16, wherein R¹⁶ is one of the following structures: [chemical structure]
